SEQUENCE LISTING (24 sequences)

SEQ ID NO: 1 - protein, 5 aa, Artificial Sequence
  Synthetic polypeptide
SYWIG

SEQ ID NO: 2 - protein, 17 aa, Artificial Sequence
  Synthetic polypeptide
IIYPGDSDTRYSPSFQG

SEQ ID NO: 3 - protein, 7 aa, Artificial Sequence
  Synthetic polypeptide
YSGSFDN

SEQ ID NO: 4 - protein, 13 aa, Artificial Sequence
  Synthetic polypeptide
SGTSSNIGSHSVN

SEQ ID NO: 5 - protein, 7 aa, Artificial Sequence
  Synthetic polypeptide
TNNQRPS

SEQ ID NO: 6 - protein, 11 aa, Artificial Sequence
  Synthetic polypeptide
AAWDGSLNGLV

SEQ ID NO: 7 - protein, 7 aa, Artificial Sequence
  Synthetic polypeptide
GYSFTSY

SEQ ID NO: 8 - protein, 6 aa, Artificial Sequence
  Synthetic polypeptide
YPGDSD

SEQ ID NO: 9 - protein, 10 aa, Artificial Sequence
  Synthetic polypeptide
GYSFTSYWIG

SEQ ID NO: 10 - protein, 10 aa, Artificial Sequence
  Synthetic polypeptide
IIYPGDSDTR

SEQ ID NO: 11 - protein, 116 aa, Artificial Sequence
  Synthetic polypeptide
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 12 - protein, 111 aa, Artificial Sequence
  Synthetic polypeptide
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 13 - protein, 5 aa, Artificial Sequence
  Synthetic polypeptide
DYYVY

SEQ ID NO: 14 - protein, 17 aa, Artificial Sequence
  Synthetic polypeptide
WINPNSGGTNYAQKFQG

SEQ ID NO: 15 - protein, 9 aa, Artificial Sequence
  Synthetic polypeptide
SQRDGYMDY

SEQ ID NO: 16 - protein, 8 aa, Artificial Sequence
  Synthetic polypeptide
TGTSSDVG

SEQ ID NO: 17 - protein, 7 aa, Artificial Sequence
  Synthetic polypeptide
EDSKRPS

SEQ ID NO: 18 - protein, 10 aa, Artificial Sequence
  Synthetic polypeptide
SSNTRSSTLV

SEQ ID NO: 19 - protein, 7 aa, Artificial Sequence
  Synthetic polypeptide
GYTFIDY

SEQ ID NO: 20 - protein, 6 aa, Artificial Sequence
  Synthetic polypeptide
NPNSGG

SEQ ID NO: 21 - protein, 10 aa, Artificial Sequence
  Synthetic polypeptide
GYTFIDYYVY

SEQ ID NO: 22 - protein, 10 aa, Artificial Sequence
  Synthetic polypeptide
WINPNSGGTN

SEQ ID NO: 23 - protein, 118 aa, Artificial Sequence
  Synthetic polypeptide
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 24 - protein, 105 aa, Artificial Sequence
  Synthetic polypeptide
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

(19) United States
(12) Patent Application Publication
(10) Pub. No.: US 2022/0054550 A1
(43) Pub. Date: Feb. 24, 2022

(54) COMBINATION THERAPIES

(71) Applicant: MEMORIAL SLOAN-KETTERING CANCER CENTER, New York, NY (US)

(72) Inventors: Eric L. Smith, New York, NY (US); Renier J. Brentjens, New York, NY (US)

(73) Assignee: MEMORIAL SLOAN-KETTERING CANCER CENTER, New York, NY (US)

(21) Appl. No.: 17/512,961

(22) Filed: Oct. 28, 2021

Related U.S. Application Data
(63) Continuation of application No. PCT/US2020/030690, filed on Apr. 30, 2020.
(60) Provisional application No. 62/840,906, filed on Apr. 30, 2019.

(51) Int. Cl. A61K 35/17; C12N 15/86; C07K 14/705; A61P 35/00

(52) U.S. Cl. CPC A61K 35/17; C12N 15/86; A61P 35/00; C07K 14/70517; C07K 14/70578; C07K 14/70521

(57) ABSTRACT

The presently disclosed subject matter provides combination therapies for treating diseases or disorders, e.g., cancers. In particular, the present disclosure provides methods of treatment comprising administering genetically engineered cells and radiation.

### 1. CROSS-REFERENCE TO RELATED APPLICATIONS

This application is a Continuation of International Patent Application No. PCT/US2020/030690, filed Apr. 30, 2020, which claims priority to U.S. Provisional Patent Application Ser. No. 62/840,906, filed Apr. 30, 2019, the contents of each of which are incorporated by reference in its entirety, and to each of which priority is claimed.

### 2. SEQUENCE LISTING

The specification further incorporates by reference the Sequence Listing submitted herewith via EFS on Oct. 28, 2021. Pursuant to 37 C.F.R. § 1.52(e)(5), the Sequence Listing text file, identified as 089333_0427_SL.txt, is 8,511 bytes and was created on Oct. 27, 2021. The Sequence Listing electronically filed herewith, does not extend beyond the scope of the specification and thus does not contain new matter.

### 3. TECHNICAL FIELD

The present disclosure relates to combination therapies for treating diseases or disorders, e.g., cancers. In particular, the present disclosure provides methods of treatment comprising administering genetically engineered cells and radiation.

### 4. BACKGROUND

Radiation therapy (XRT) works locally though DNA damage of tumor cells and can induce a systemic abscopal effect; that is, it can provide an immune mediated anti-tumor response outside the radiation field. This response is facilitated by release of neo-antigens that generate and shape a systemic TCR-mediated anti-tumor immune response (Sridharan et al., Br J Cancer (2016); 115:252-60; Tang et al., Cancer Immunol Res (2014); 2:831-8). Checkpoint blockade synergizes with concurrent XRT, likely because of this increased T-cell exposure to neo-antigens et al., Oncoimmunology (2014); 3:e28780; Postow et al., N Engl J Med. (2012); 366:925-31; Park et al., Cancer Immunol Res (2015); 3:610-9; Victor et al., Nature (2015); 520:373-7).

Various immunotherapy and/or cell therapy methods are available for treating diseases and conditions. For example, adoptive cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be effective in the treatment of cancer and other diseases and disorders. Alternative methods are needed, for example, to provide increased efficacy and/or reduced cytokine release syndrome in certain patient populations and/or for treating certain diseases or conditions. Among the provided embodiments are methods and uses that address such needs.

### 5. SUMMARY OF THE INVENTION

Provided are methods and compositions for treatment of subjects having or suspected of having a disease or condition, such as cancer or other proliferative disorder. In certain embodiments, the method includes (a) administering to a subject having a disease or condition a dose of cells expressing a recombinant receptor that binds to an antigen; and (b) administering to said subject radiation, wherein initiation of administration of the radiation is no later than about two weeks after administration of the recombinant receptor-expressing cells.

In certain embodiments, initiation of administration of the radiation is no later than about one week after administration of the recombinant receptor-expressing cells. In certain embodiments, initiation of administration of the radiation is between about 5 days and about 10 days after administration of the recombinant receptor-expressing cells. In certain embodiments, the subject has not relapsed at the time of or immediately prior to initiation of administration of the radiation.

In certain embodiments, the disease or condition is a tumor or a cancer. The disease or condition can be selected from the group consisting of blood cancers, B cell malignancies, colon cancer, lung cancer, liver cancer, breast cancer, prostate cancer, ovarian cancer, skin cancer, melanoma, bone cancer, brain cancer, ovarian cancer, epithelial cancers, renal cell carcinoma, pancreatic adenocarcinoma, cervical carcinoma, colorectal cancer, glioblastoma, neuroblastoma, Ewing sarcoma, medulloblastoma, osteosarcoma, synovial sarcoma, mesothelioma, and combinations thereof. In certain embodiments, the blood cancer is selected from the group consisting of leukemia, lymphoma, chronic lymphocytic leukemia (CLL), acute-lymphoblastic leukemia (ALL), Hodgkin Lymphoma, non-Hodgkin's lymphoma, Waldenstrom's Macroglobulinemia, acute myeloid leukemia, multiple myeloma, mantle cell lymphoma, and indolent B cell lymphoma. In certain embodiments, the disease or condition is multiple myeloma.

The antigen can be a tumor antigen or a pathogen antigen. In certain embodiments, the antigen is a tumor antigen. The tumor antigen can be selected from the group consisting of BCMA, GPRCSD, FcRL5, orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, OEPHa2, Erb-B2, Erb-B3, Erb-B4, FBP, fetal acethycholine e receptor, GD2, GD3, HMW-MAA, IL-22R-alpha, IL-13R-alpha2, KDR, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, and MAGE A3, CE7, Wilms Tumor 1 (WT-1), a cyclin, and biotinylated molecules. In certain embodiments, the tumor antigen is BCMA.

In certain embodiments, the recombinant receptor is a T cell receptor (TCR) or a functional non-T cell receptor. In certain embodiments, the recombinant receptor is a chimeric antigen receptor (CAR).

The CAR can include an extracellular antigen-binding domain that specifically binds to the antigen and an intracellular signaling domain. In certain embodiments, the intracellular signaling domain comprises an intracellular domain of a CD3-zeta (CD3ζ) chain. The intracellular signaling domain can further include a costimulatory signaling region. The costimulatory signaling region can include a signaling domain of CD28 or a portion thereof, a signaling domain of 4-1BB or a portion thereof, a signaling domain of OX40 or a portion thereof, a signaling domain of ICOS or a portion thereof, a signaling domain of DAP-10 or a portion thereof, or a combination thereof. In certain embodiments, the costimulatory signaling region comprises a signaling domain of 4-1BB or a portion thereof.

The CAR can further include a transmembrane domain. The transmembrane domain can include a transmembrane domain of CD8 or a portion thereof, or a transmembrane domain of CD28 or a portion thereof. In certain embodiments, the transmembrane domain comprises a transmembrane domain of a CD8 or a portion thereof.

In certain embodiments, the extracellular antigen-binding domain comprises a scFv. In certain embodiments, the extracellular antigen-binding domain comprises: a heavy chain variable region (“V_{H}”) CDR1 comprising the amino acid sequence set forth in SEQ ID NO: 1; a V_{H} CDR2 comprising the amino acid sequence set forth in SEQ ID NO: 2; a V_{H} CDR3 comprising the amino acid sequence set forth in SEQ ID NO: 3; a light chain variable region (“V_{L}”) CDR1 comprising the amino acid sequence set forth in SEQ ID NO: 4; a V_{L} CDR2 comprising the amino acid sequence set forth in SEQ ID NO: 5; and a V_{L} CDR3 comprising the amino acid sequence set forth in SEQ ID NO: 6.

In certain embodiments, the cell is a T cell. The T cell can be selected from the group consisting of a cytotoxic T lymphocyte (CTL), a regulatory T cell, a tumor-infiltrating lymphocyte (TIL), and a Natural Killer T (NKT) cell. The cell can be autologous or allogenic to the subject.

In certain embodiments, the dose of cells comprises cells in an amount sufficient for reduction in burden of a disease or condition in the subject. In certain embodiments, the method includes administering to the subject a consecutive dose of the recombinant receptor-expressing cells after administration of a first dose of the recombinant receptor-expressing cells.

The radiation can be selected from the group consisting of external beam radiation, a radiopharmaceutical agent, and a combination thereof. In certain embodiments, the radiation is external beam radiation. In certain embodiments, a total of at least about 10 Gy of radiation is administered to a lesion site of the subject. In certain embodiments, a total of between about 10 Gy and about 30 Gy of radiation is administered to a lesion site of the subject. In certain embodiments, a total of about 20 Gy of radiation is administered to a lesion site of the subject.

In certain embodiments, administration of the radiation and administration of the recombinant receptor-expressing cells provide a synergistic abscopal effect. In certain embodiments, administration of the radiation and administration of the recombinant receptor-expressing cells provide delayed or reduced CRS-like response. In certain embodiments, administration of the radiation and administration of the recombinant receptor-expressing cells provide systemic expansion of new T-cell receptor (TCR) clone.

The present discourse also provides radiation and cells expressing a recombinant receptor that binds to an antigen are for use in a therapy, wherein initiation of administration of the radiation is no later than about two weeks after administration of the recombinant receptor-expressing cells.

### 6. BRIEF DESCRIPTION OF THE DRAWINGS

FIGS. 1A-1F depict BCMA-targeted CAR T-cell therapy followed by radiation therapy (XRT) led to clinical response, expansion of TCR clonality, and CRS-like findings after XRT. The subject received conditioning therapy with cyclophosphamide and fludarabine followed by CAR T cells on day 0. Radiation therapy (XRT) took place over 10 fractions between day 6 and day 20 (box). FIG. 1A shows pre-treatment PET/CT scan showing extensive intra-osseous and extra-osseous FDG-avid disease including soft tissue and pleural-based masses. FIG. 1B shows decrease in M-spike commencing during XRT. FIG. 1C shows PET/CT 8 weeks post-therapy demonstrating resolution of MM lesions. FIG. 1D shows production of IL6 and CRP (pro-inflammatory markers associated with active CAR T-cell function) peaked after the conclusion of XRT. FIG. 1E shows daily maximum temperature curve revealed a fever at the time of peak IL6 and CRP. FIG. 1F shows TCR clonality analysis demonstrating expansion of novel TCR clones. The subset of TCRs comprising newly expanding clones are shown over time.

FIGS. 2A and 2B depict local early response to radiation therapy. FIG. 2A shows MRIs pre- (lef) and 4 weeks post- (right) CAR T cell therapy (1 week post-conclusion of radiation therapy). FIG. 2B shows radiation fields. Conventionally fractioned radiation therapy was delivered to the thoracic spine (T1 to T8) over 5 days, followed by to the whole brain to C2 over 5 days. The total dose was 2000 cGy in 5 fractions to each site.

FIGS. 3A-3C depict additional inflammatory markers elevated in response to CAR T cell therapy plus radiation therapy. FIG. 3A shows Ferritin. FIG. 3B shows D-dimer. FIG. 3C shows IL-10.

FIG. 4 depicts CAR T cells expanded and maintained persistence after the initiation of high dose steroid taper and including through the period of radiation therapy. CAR transgene as detected by PCR from peripheral blood.

FIG. 5 depicts persistence of CAR T cells around the time of expansion of TCR clonality and CRS-like findings after XRT. CAR T cells in the patient's blood were assessed at 5 weeks after CAR T-cell therapy by flow cytometry with a fluorophore-labeled antibody recognizing the surrogate transduction marker that is additionally expressed by the CAR vector. Flow cytometry of peripheral blood mononuclear cells was gated on viable CD3⁺ cells. The red box outlines cells positive for the transduction marker (9.3% of circulating T cells). Note that these persistent gene-modified T cells were predominantly CD8⁺.

FIG. 6 depicts TCR repertoire of bone marrow mirrored that of peripheral blood. The TCR repertoire of bone marrow and peripheral blood mononuclear cells from the same time point was evaluated. TCR diversity was compared by Morisita's Overlap Index (C_{D}).

FIG. 7 depicts addition of radiation therapy to CAR T-cell therapy increased TCR repertoire diversity over time. Heatmap of Morisita's Overlap Indices (C_{D}) displays the degree of similarity between TCR repertoire from peripheral blood samples over time. The index ranges from 0 (no overlap of TCR clonotypes) to 1 (all TCR clonotypes occur in the same proportions in both samples).

FIG. 8 depicts highly expressed baseline TCR clones did not expand after CAR T cell administration and radiation therapy. The frequency of the subset of TCRs comprising TCR clones with baseline >2% frequency is shown over time.

### 7. DETAILED DESCRIPTION OF THE INVENTION

The presently disclosed subject matter provides methods of treatments involving the administration of genetically engineered cells and radiation to subjects having a disease or condition. The cells are engineered to express one or more recombinant receptor, for example a chimeric antigen receptor (CAR).

The section headings used herein are for organizational purposes only and are not to be construed as limiting the subject matter described.

For purposes of clarity of disclosure and not by way of limitation, the detailed description is divided into the following subsections:

7.1. Definitions;

7.2. Methods for Treatments by Using Genetically Engineered Cells and Radiation;

7.3. Recombinant Receptors Expressed by the Cells;

7.4. Genetically Engineered Cells and Methods of Producing Cells;

7.5. Compositions and Formulations of Genetically Engineered Cells;

7.6. Dosing of the Genetically Engineered Cells;

7.7. Radiation Therapy;

7.8. Administration of Radiation Therapy;

7.9. Reduction in Disease Burden, Efficacy and Survival; and

7.10. Kits

### 7.1. DEFINITIONS

Unless defined otherwise, all technical and scientific terms used herein have the meaning commonly understood by a person skilled in the art.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant CL region. The light chain constant region is comprised of one domain, CL. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242). In certain embodiments, the CDRs are delineated using the Chothia numbering system (Chothia et al., J Mol Biol. (1987) 196:901-17). In certain embodiments, the CDRs are delineated using the AbM numbering system (Abhinandan et al., Mol. Immunol. 2008, 45, 3832-3839). In certain embodiments, the CDRs regions are delineated using the IMGT numbering system (accessible at http://www.imgt.org/IIVIGTScientificChart/Numbering/IMGTIGVLsuperfamily.html, http://www.imgt.org/IMGTindex/numbering.php).

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the Cterminus of the V_{L}, or the C-terminus of the V_{H} with the N-terminus of the V_{L}. The linker is usually rich in glycine for flexibility, as well as serine or threonine for solubility.

As used herein, the term “linker” means a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). Despite removal of the constant regions and the introduction of a linker, scFv proteins retain the specificity of the original immunoglobulin. Single chain Fv polypeptide antibodies can be expressed from a nucleic acid including V_{H}- and V_{L} encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors. In certain embodiments, a vector refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the term “expression vector” refers to a recombinant nucleic acid sequence, i.e. recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

By “substantially identical” or “substantially homologous” is meant a polypeptide or a nucleic acid molecule exhibiting at least about 50% homologous or identical to a reference amino acid sequence (for example, any one of the amino acid sequences described herein) or a nucleotide acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the amino acid sequence or the nucleotide sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “disease” is meant any condition, disease or disorder that damages or interferes with the normal function of a cell, tissue, or organ, e.g., neoplasia, and pathogen infection of cell.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or a set of antigenic determinants present on a cell.

By “neoplasm” is meant a disease characterized by the pathological proliferation of a cell or tissue and its subsequent migration to or invasion of other tissues or organs. The growth of neoplasm is typically uncontrolled and progressive, and occurs under conditions that would not elicit, or would cause cessation of, multiplication of normal cells. Neoplasm can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells).

By “recognize” is meant selectively binds to a target. A T cell that recognizes a tumor can expresses a CAR that binds to a tumor antigen.

By “specifically binds” is meant a polypeptide or a fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein includes a tumor-specific antigen (TSA), which is present only on tumor cells and not on any other cell, and tumor-associated antigen (TAA), which is present on some tumor cells and also some normal cells. In certain embodiments, the tumor antigen is a TSA. In certain embodiments, the tumor antigen is uniquely or differentially expressed on a tumor cell compared to a normal or non-neoplastic cell. In certain embodiments, a tumor antigen includes any polypeptide expressed by a tumor that is capable of activating or inducing an immune response via an antigen recognizing receptor (e.g., mesothelin) or capable of suppressing an immune response via receptor-ligand binding (e.g., CD47, PD-L1/L2, B7.1/2).

The terms “comprises”, “comprising”, and are intended to have the broad meaning ascribed to them in U.S. Patent Law and can mean “includes”, “including” and the like.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In certain embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In certain embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. In certain embodiments, the primate is a monkey or an ape. The subject can be male or female and can be any suitable age, including infant, juvenile, adolescent, adult, and geriatric subjects. In certain embodiments, the subject is a non-primate mammal, such as a rodent.

As used herein, the singular forms “a,” “an,” and “the” include plural referents unless the context clearly dictates otherwise. For example, “a” or “an” means “at least one” or “one or more.”

As used herein, a “composition” refers to any mixture of two or more products, substances, or compounds, including cells. It may be a solution, a suspension, liquid, powder, a paste, aqueous, non-aqueous or any combination thereof.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

### 7.2. METHODS FOR TREATMENTS BY USING GENETICALLY ENGINEERED CELLS AND RADIATION

The presently disclosed subject matter is based on the discovery that genetically engineered cells (e.g., chimeric antigen receptor (CAR)-expressing T cells) can be used in combination with a radiation source, such as external beam irradiation or a radioisotope, such as a radiopharmaceutical, to provide therapeutically effective anticancer effects.

Furthermore, an unexpected synergistic effect between the genetically engineered cells and the radiation source results in an enhanced or synergistic therapeutic effect, wherein the combined effect is greater than the additive effect resulting from administration of the cells and radiation each at a therapeutic dose. These observations support that genetically engineered cells (e.g., chimeric CAR-expressing T cells) can be used in combination with radiotherapy for treating diseases or disorders (e.g., cancers).

The present disclosure is at least based on the observation that the combination of a first treatment procedure that includes administration of an effective amount of genetically engineered cells (e.g., CAR-expressing T cells), as described herein, and a second treatment procedure using radiation treatment, as described herein, to a subject in need thereof can provide a) a synergistic abscopal-like response, therapeutically effective anti-cancer effects, delayed or reduced CRS-like response, and/or systemic expansion of new T-cell receptor (TCR) clone (e.g., in peripheral blood).

Provided are methods for treating diseases or conditions, including various cancers and tumors. In certain embodiments, the method includes administering (a) radiation and (b) cells expressing recombinant receptors designed to recognize and/or specifically bind to a molecule (e.g., an antigen) associated with the disease or condition to be treated and result in a response, such as an immune response against such molecule upon binding to such molecule. The recombinant receptors include chimeric receptors, e.g., chimeric antigen receptors (CARs), and other transgenic antigen receptors including transgenic T cell receptors (TCRs).

In certain embodiments, the methods are advantageous in their ability to treat subjects having certain diseases or conditions such as cancers (e.g., multiple myeloma) by initiating the radiation therapy soon after the subject has received the cells expressing the recombinant receptor (referred to as “the recombinant receptor-expressing cells”). For example, in certain embodiments, the method comprises initiating the radiation therapy no later than about two weeks after administration of the recombinant receptor-expressing cells. In certain embodiments, the method comprises initiating the radiation therapy no later than about one week after administration of the recombinant receptor-expressing cells. In certain embodiments, the method comprises initiating the radiation therapy between about 5 days and about 10 days after administration of the recombinant receptor-expressing cells. In certain embodiments, the method comprises initiating the radiation therapy about 5 days after administration of the recombinant receptor-expressing cells. In certain embodiments, the method comprises initiating the radiation therapy 6 days after administration of the recombinant receptor-expressing cells.

In certain embodiments, the subject has not relapsed at the time of or immediately prior to initiation of the radiation therapy.

In certain embodiments, the method includes administration of the recombinant receptor-expressing cells or a composition comprising the cells to a subject, tissue, or cell, such as one having, at risk for, or suspected of having the disease, condition or disorder. In certain embodiments, the recombinant receptor-expressing cells or compositions comprising thereof are administered to a subject having the particular disease or condition to be treated, e.g., via adoptive cell therapy, such as adoptive T cell therapy. In certain embodiments, the recombinant receptor-expressing cells or compositions comprising thereof are administered to the subject, such as a subject having or at risk for the disease or condition, ameliorate one or more symptom of the disease or condition.

The disease or condition that is treated is generally one with which expression of the target antigen is associated and/or involved in the etiology thereof, e.g. in which the antigen causes, exacerbates or otherwise is involved in such disease, condition, or disorder or is simply a marker of or is overexpressed or uniquely expressed on cells of the disease or disorder. Exemplary diseases and conditions can include, but are not limited to, diseases or conditions associated with malignancy or transformation of cells (e.g. cancer). Exemplary antigens, which include antigens associated with various diseases and conditions that can be treated, are described above. In certain embodiments, the CAR or TCR specifically binds to an antigen associated with the disease or condition.

Among the diseases, conditions, and disorders are tumors, including solid tumors, hematologic malignancies (e.g., blood cancers), and melanomas, and including localized and metastatic tumors. In certain embodiments, the subject has an infectious disease, such as infection with a virus or other pathogen, e.g., HIV, HCV, HBV, CMV, and parasitic disease.

In certain embodiments, the disease or condition is a tumor, cancer, malignancy, neoplasm, or other proliferative disease or disorder. Such diseases include but are not limited to, blood cancers (including, but not limited to, leukemia, chronic lymphocytic leukemia (CLL), acute-lymphoblastic leukemia (ALL), non-Hodgkin's lymphoma (e.g., refractory follicular lymphoma), Waldenstrom's Macroglobulinemia, Hodgkin's lymphoma, acute myeloid leukemia, multiple myeloma, mantle cell lymphoma, and indolent B cell lymphoma), B cell malignancies, colon cancer, lung cancer, liver cancer, breast cancer, prostate cancer, ovarian cancer, skin cancer, melanoma, bone cancer, brain cancer, ovarian cancer, epithelial cancers, renal cell carcinoma, pancreatic adenocarcinoma, cervical carcinoma, colorectal cancer, glioblastoma, neuroblastoma, Ewing sarcoma, medulloblastoma, osteosarcoma, synovial sarcoma, and/or mesothelioma. In certain embodiments, the subject has blood cancer. In certain embodiments, the disease is multiple myeloma.

In certain embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In certain embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In certain embodiments, the antigen associated with the disease or disorder is selected from the group consisting of BCMA, orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, OEPHa2, Erb-B2, Erb-B3, Erb-B4, FBP, fetal acethycholine e receptor, GD2, GD3, HMW-MAA, IL-22R-alpha, IL-13R-alpha2, KDR, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, and MAGE A3, CE7, Wilms Tumor 1 (WT-1), a cyclin (e.g., cyclin A1 (CCNA1)), biotinylated molecules, molecules expressed by HIV, HCV, HBV, and other pathogens. In certain embodiments, the antigen is BCMA.

In certain embodiments, the provided methods can enhance overall survival of subjects in which subjects exhibit a particular level of disease burden at the time of treatment such as morphologic or minimal disease.

### 7.3. RECOMBINANT RECEPTORS EXPRESSED BY THE CELLS

In certain embodiments, the cells for use in or administered in connection with the provided methods contain or are engineered to include a recombinant receptor (or an engineered receptor), e.g., an engineered antigen receptor, such as a chimeric antigen receptor (CAR), or a T cell receptor (TCR). Also provided are populations of such cells, compositions containing such cells and/or enriched for such cells, such as in which cells of a certain type such as T cells or CD8⁺ or CD4⁺ cells are enriched or selected. Among the compositions are pharmaceutical compositions and formulations for administration, such as for adoptive cell therapy. Also provided are therapeutic methods for administering the cells and compositions to subjects, e.g., patients, in accord with the provided methods.

In certain embodiments, the cells include one or more nucleic acids introduced via genetic engineering, and thereby express recombinant or genetically engineered products of such nucleic acids. In certain embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

The cells generally express recombinant receptors, such as antigen receptors including functional non-TCR antigen receptors, e.g., chimeric antigen receptors (CARs), and other antigen-binding receptors such as transgenic T cell receptors (TCRs).

### 7.3.1. CHIMERIC ANTIGEN RECEPTORS (CARS

In certain embodiments of the provided methods and uses, the recombinant receptor is a chimeric antigen receptor (CAR), which comprises an extracellular antigen-binding domain that provides specificity for an antigen (e.g., a tumor antigen) and an intracellular signaling domain. In certain embodiments, the CAR comprises a transmembrane domain. In certain embodiments, the intracellular signaling domain is an activating intracellular domain portion, such as a T cell activating domain, providing a primary activation signal. In certain embodiments, the intracellular signaling domain includes a costimulatory signaling region to facilitate effector functions. In certain embodiments, the recombinant receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In certain embodiments, the recombinant receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

The recombinant receptors, such as CARs, generally include an extracellular antigen-binding domain, such as a portion of an antibody molecule, generally a variable heavy (V_{H}) chain region and/or variable light (V_{L}) chain region of the antibody. In certain embodiments, the extracellular antigen-binding domain comprises a scFv. The scFv can be a human, murine or humanized scFv. In certain embodiments, the scFv is a human scFv. In certain embodiments, the extracellular antigen-binding domain comprises a Fab, which is optionally crosslinked. In certain embodiments, the extracellular antigen-binding domain comprises a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. The scFv can be derived from a mouse bearing human V_{L} and/or V_{H} genes. The scFv can also be substituted with a camelid Heavy chain (e.g., VHH, from camel, lama, etc.) or a partial natural ligand for a cell surface receptor.

In certain embodiments, the antigen targeted by the receptor is a polypeptide. In certain embodiments, it is a carbohydrate or other molecule. In certain embodiments, the antigen is selectively expressed or overexpressed on cells of the disease or condition, e.g., the tumor or pathogenic cells, as compared to normal or non-targeted cells or tissues. In certain embodiments, the antigen is expressed on normal cells and/or is expressed on the engineered cells. In certain embodiments, the antigen is a tumor antigen or a pathogen antigen.

In certain embodiments, antigens targeted by the receptors include, but not limited to, BCMA, G-protein coupled receptor (e.g., a G-protein coupled receptor family C group 5 member D (GPRCSD)), Fc Receptor-like 5 (FcRL5), orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, OEPHa2, Erb-B2, Erb-B3, Erb-B4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, and MAGE A3, CE7, Wilms Tumor 1 (WT-1), a cyclin (e.g., cyclin A1 (CCNA1)), and biotinylated molecules. In certain embodiments, antigens targeted by the receptors include, but not limited to, molecules expressed by HIV, HCV, HBV, and other pathogens.

In certain embodiments, the CAR binds to a pathogen-specific antigen. In certain embodiments, the CAR is specific for viral antigens (such as HIV, HCV, HBV, etc.), bacterial antigens, and/or parasitic antigens.

In certain embodiments, the recombinant receptor (e.g., a CAR) targets BCMA (e.g., human BCMA). In certain embodiments, the cells of the presently disclosed subject matter express a BCMA-targeted CAR disclosed in the International Patent Application Publication No. WO2016/090320, which is incorporated by reference in its entirety. In certain embodiments, the cells of the presently disclosed subject matter express the BCMA-targeted CAR disclosed in Smith et al., Molecular Therapy (2018); 26(6):1447-1456), which is incorporated by reference in its entirety. In certain embodiments, the extracellular antigen-binding domain of the BCMA-targeted CAR comprises an antigen-binding fragment (e.g., scFv) disclosed in WO2016/090320 and WO2016/090327, which are incorporated by reference in their entireties.

In certain embodiments, the CAR includes an extracellular antigen-binding domain that includes an antibody or a fragment thereof. In certain embodiments, the CAR includes an extracellular antigen-binding domain and an intracellular signaling domain. In certain embodiments, the extracellular antigen-binding domain includes a scFv.

In certain embodiments, the CAR comprises an extracellular antigen-binding domain that comprises: a heavy chain variable region (“V_{H}”) CDR1 comprising the amino acid sequence set forth in SEQ ID NO: 1; a V_{H} CDR2 comprising the amino acid sequence set forth in SEQ ID NO: 2; a V_{H} CDR3 comprising the amino acid sequence set forth in SEQ ID NO: 3; a light chain variable region (“V_{L}”) CDR1 comprising the amino acid sequence set forth in SEQ ID NO: 4; a V_{L} CDR2 comprising the amino acid sequence set forth in SEQ ID NO: 5; and a V_{L} CDR3 comprising the amino acid sequence set forth in SEQ ID NO: 6. In certain embodiments, the CDRs are identified according to the Kabat numbering system. SEQ ID Nos: 1-6 are provided below.

[SEQ ID NO: 1]
SYWIG
[SEQ ID NO: 2]
IIYPGDSDTRYSPSFQG
[SEQ ID NO: 3]
YSGSFDN
[SEQ ID NO: 4]
SGTSSNIGSHSVN
[SEQ ID NO: 5]
TNNQRPS
[SEQ ID NO: 6]
AAWDGSLNGLV

In certain embodiments, the CAR comprises an extracellular antigen-binding domain that comprises: a V_{H} CDR1 comprising the amino acid sequence set forth in SEQ ID NO: 7; a V_{H} CDR2 comprising the amino acid sequence set forth in SEQ ID NO: 8; a V_{H} CDR3 comprising the amino acid sequence set forth in SEQ ID NO: 3; a V_{L} CDR1 comprising the amino acid sequence set forth in SEQ ID NO: 4; a V_{L} CDR2 comprising the amino acid sequence set forth in SEQ ID NO: 5; and a V_{L} CDR3 comprising the amino acid sequence set forth in SEQ ID NO: 6. In certain embodiments, the CDRs are identified according to the Chothia numbering system. SEQ ID Nos: 7 and 8 are provided below.

[SEQ ID NO: 7]
GYSFTSY
[SEQ ID NO: 8]
YPGDSD

In certain embodiments, the CAR comprises an extracellular antigen-binding domain that comprises: a V_{H} CDR1 comprising the amino acid sequence set forth in SEQ ID NO: 9; a V_{H} CDR2 comprising the amino acid sequence set forth in SEQ ID NO: 10; a V_{H} CDR3 comprising the amino acid sequence set forth in SEQ ID NO: 3; a V_{L} CDR1 comprising the amino acid sequence set forth in SEQ ID NO: 4; a V_{L} CDR2 comprising the amino acid sequence set forth in SEQ ID NO: 5; and a V_{L} CDR3 comprising the amino acid sequence set forth in SEQ ID NO: 6. In certain embodiments, the CDRs are identified according to the AbM numbering system. SEQ ID Nos: 9 and 10 are provided below.

[SEQ ID NO: 9]
GYSFTSYWIG
[SEQ ID NO: 10]
IIYPGDSDTR

In certain embodiments, the CAR comprises an extracellular antigen-binding domain that comprises: a V_{H} comprising an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% homologous or identical to the amino acid sequence set forth in SEQ ID NO: 11. In certain embodiments, the CAR comprises an extracellular antigen-binding domain that comprises: a V_{H} comprising the amino acid sequence set forth in SEQ ID NO: 11. SEQ ID NO: 11 is provided below.

[SEQ ID NO: 11]
EVQLVQSGAEVKKPGESLKISCKGSGYSFTSYWIGWVRQMPGKGLEWMGI
IYPGDSDTRYSPSFQGHVTISADKSISTAYLQWSSLKASDTAMYYCARYS
GSFDNWGQGTLVTVSS

In certain embodiments, the CAR comprises an extracellular antigen-binding domain that comprises: a V_{L} comprising an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% homologous or identical to the amino acid sequence set forth in SEQ ID NO: 12. In certain embodiments, the CAR comprises an extracellular antigen-binding domain that comprises: a V_{H} comprising the amino acid sequence set forth in SEQ ID NO: 12. SEQ ID NO: 12 is provided below.

[SEQ ID NO: 12]
SYELTQPPSASGTPGQRVTMSCSGTSSNIGSHSVNWYQQLPGTAPKLLIY
TNNQRPSGVPDRFSGSKSGTSASLAISGLQSEDEADYYCAAWDGSLNGLV
FGGGTKLTVLG

In certain embodiments, the CAR comprises an extracellular antigen-binding domain that comprises: a V_{H} CDR1 comprising the amino acid sequence set forth in SEQ ID NO: 13; a V_{H} CDR2 comprising the amino acid sequence set forth in SEQ ID NO: 14; a V_{H} CDR3 comprising the amino acid sequence set forth in SEQ ID NO: 15; a V_{L} CDR1 comprising the amino acid sequence set forth in SEQ ID NO: 16; a V_{L} CDR2 comprising the amino acid sequence set forth in SEQ ID NO: 17; and a V_{L} CDR3 comprising the amino acid sequence set forth in SEQ ID NO: 18. In certain embodiments, the CDRs are identified according to the Kabat numbering system. SEQ ID Nos: 13-18 are provided below.

[SEQ ID NO: 13]
DYYVY
[SEQ ID NO: 14]
WINPNSGGTNYAQKFQG
[SEQ ID NO: 15]
SQRDGYMDY
[SEQ ID NO: 16]
TGTSSDVG
[SEQ ID NO: 17]
EDSKRPS
[SEQ ID NO: 18]
SSNTRSSTLV

In certain embodiments, the CAR comprises an extracellular antigen-binding domain that comprises: a V_{H} CDR1 comprising the amino acid sequence set forth in SEQ ID NO: 19; a V_{H} CDR2 comprising the amino acid sequence set forth in SEQ ID NO: 20; a V_{H} CDR3 comprising the amino acid sequence set forth in SEQ ID NO: 15; a V_{L} CDR1 comprising the amino acid sequence set forth in SEQ ID NO: 16; a V_{L} CDR2 comprising the amino acid sequence set forth in SEQ ID NO: 17; and a V_{L} CDR3 comprising the amino acid sequence set forth in SEQ ID NO: 18. In certain embodiments, the CDRs are identified according to the Chothia numbering system. SEQ ID Nos: 19 and 20 are provided below.

[SEQ ID NO: 19]
GYTFIDY
[SEQ ID NO: 20]
NPNSGG

In certain embodiments, the CAR comprises an extracellular antigen-binding domain that comprises: a V_{H} CDR1 comprising the amino acid sequence set forth in SEQ ID NO: 21; a V_{H} CDR2 comprising the amino acid sequence set forth in SEQ ID NO: 22; a V_{H} CDR3 comprising the amino acid sequence set forth in SEQ ID NO: 15; a V_{L} CDR1 comprising the amino acid sequence set forth in SEQ ID NO: 16; a V_{L} CDR2 comprising the amino acid sequence set forth in SEQ ID NO: 17; and a V_{L} CDR3 comprising the amino acid sequence set forth in SEQ ID NO: 18. In certain embodiments, the CDRs are identified according to the AbM numbering system. SEQ ID Nos: 21 and 22 are provided below.

[SEQ ID NO: 21]
GYTFIDYYVY
[SEQ ID NO: 22]
WINPNSGGTN

In certain embodiments, the CAR comprises an extracellular antigen-binding domain that comprises: a V_{H} comprising an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% homologous or identical to the amino acid sequence set forth in SEQ ID NO: 23. In certain embodiments, the CAR comprises an extracellular antigen-binding domain that comprises: a V_{H} comprising the amino acid sequence set forth in SEQ ID NO: 23. SEQ ID NO: 23 is provided below.

[SEQ ID NO: 23]
EVQLVQSGAEMKKPGASLKLSCKASGYTFIDYYVYWMRQAPGQGLESMGW
INPNSGGTNYAQKFQGRVTMTRDTSISTAYMELSRLRSDDTAMYYCARSQ
RDGYMDYWGQGTLVTVSS

In certain embodiments, the CAR comprises an extracellular antigen-binding domain that comprises: a V_{L} comprising an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% homologous or identical to the amino acid sequence set forth in SEQ ID NO: 24. In certain embodiments, the CAR comprises an extracellular antigen-binding domain that comprises: a V_{H} comprising the amino acid sequence set forth in SEQ ID NO: 24. SEQ ID NO: 24 is provided below.

[SEQ ID NO: 24]
QSALTQPASVSASPGQSIAISCTGTSSDVGWYQQHPGKAPKLMIYEDSKR
PSGVSNRFSGSKSGNTASLTISGLQAEDEADYYCSSNTRSSTLVFGGGTK
LTVLG

In certain embodiments, the antibody portion of the recombinant receptor, e.g., CAR, further includes at least a portion of an immunoglobulin constant region, such as a hinge region, e.g., an IgG4 hinge region, and/or a CH1/CL and/or Fc region. In certain embodiments, the constant region or portion is of a human IgG, such as IgG4 or IgG1. In certain embodiments, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

In certain embodiments, the intracellular signaling domain is linked directly or indirectly to the extracellular antigen-binding domain. In certain embodiments, the CAR includes a transmembrane domain linking the extracellular antigen-binding domain and the intracellular signaling domain. In certain embodiments, the intracellular signaling domain comprises an ITAM. For example, in certain embodiments, the extracellular antigen-binding domain is linked to one or more intracellular signaling components, such as signaling components that mimic activation through an antigen receptor complex, such as a TCR complex, in the case of a CAR, and/or signal via another cell surface receptor.

In certain embodiments, a transmembrane domain that naturally is associated with one of the domains in the receptor, e.g., CAR, is used. In certain embodiments, the transmembrane domain is selected or modified by amino acid substitution to avoid binding of such domains to the transmembrane domains of the same or different surface membrane proteins to minimize interactions with other members of the receptor complex.

In certain embodiments, the transmembrane domain is derived either from a natural or from a synthetic source. Where the source is natural, the domain, in certain embodiments is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, CD 154. Alternatively, in certain embodiments, the transmembrane domain is synthetic. In certain embodiments, the synthetic transmembrane domain comprises predominantly hydrophobic residues such as leucine and valine. In certain embodiments, a triplet of phenylalanine, tryptophan and valine is be found at each end of a synthetic transmembrane domain. In certain embodiments, the linkage is by linkers, spacers, and/or transmembrane domain(s). In certain embodiments, the transmembrane domain includes a transmembrane portion of CD28. In certain embodiments, the transmembrane domain includes a transmembrane portion of CD8.

In certain embodiments, the extracellular antigen-binding domain and transmembrane domain can be linked directly or indirectly. In certain embodiments, the extracellular antigen-binding domain and transmembrane are linked by a spacer, such as any described herein. In certain embodiments, the recombinant receptor (e.g., CAR) includes extracellular portion of the molecule from which the transmembrane domain is derived, such as a CD28 extracellular portion, or a CD8 extracellular portion.

Among the intracellular signaling domains are those that mimic or approximate a signal through a natural antigen receptor, a signal through such a receptor in combination with a costimulatory receptor, and/or a signal through a costimulatory receptor alone. In certain embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

In certain embodiments, T cell activation is described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In certain embodiments, the CAR includes one or both of such signaling components.

The recombinant receptor, e.g., the CAR, generally includes at least one intracellular signaling component. In certain embodiments, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In certain embodiments, cytoplasmic signaling molecule(s) in the CAR contain(s) a cytoplasmic signaling domain, portion thereof, or sequence derived from CD3 zeta.

In certain embodiments, the recombinant receptor includes an intracellular component of a TCR complex, such as a TCR CD3 chain that mediates T-cell activation and cytotoxicity, e.g., CD3 zeta chain. Thus, in certain embodiments, the antigen-binding portion is linked to one or more cell signaling modules. In certain embodiments, the cell signaling modules include a CD3 transmembrane domain, a CD3 intracellular signaling domain, and/or other CD transmembrane domains. In certain embodiments, the recombinant receptor, e.g., CAR, further includes a portion of one or more additional molecules such as Fc receptor γ, CD8, CD4, CD25, or CD16. For example, in certain embodiments, the CAR or other chimeric receptor includes a chimeric molecule between CD3-zeta (CD3-ζ) or Fc receptor γ and CD8, CD4, CD25 or CD16.

In certain embodiments, upon ligation of the CAR or other recombinant receptor, the cytoplasmic domain or intracellular signaling domain of the receptor activates at least one of the normal effector functions or responses of the immune cell, e.g., T cell engineered to express the CAR. For example, in certain embodiments, the CAR induces a function of a T cell such as cytolytic activity or T-helper activity, such as secretion of cytokines or other factors. In certain embodiments, a truncated portion of an intracellular signaling domain of an antigen receptor component or costimulatory molecule is used in place of an intact immunostimulatory chain, for example, if it transduces the effector function signal. In certain embodiments, the intracellular signaling domain or domains include the cytoplasmic sequences of the T cell receptor (TCR), and in certain embodiments also those of co-receptors that in the natural context act in concert with such receptors to initiate signal transduction following antigen receptor engagement.

In the context of a natural TCR, full activation generally requires not only signaling through the TCR, but also a costimulatory signal. Thus, in certain embodiments, to promote full activation, a component for generating secondary or co-stimulatory signal is also included in the CAR. In certain embodiments, the CAR does not include a component for generating a costimulatory signal. In certain embodiments, an additional CAR is expressed in the same cell and provides the component for generating the secondary or costimulatory signal.

In certain embodiments, the CAR includes both the activating and costimulatory components. In certain embodiments, the intracellular signaling domain of the CAR comprises at least one co-stimulatory signaling region. In certain embodiments, the at least one co-stimulatory signaling region comprises an intracellular domain of a costimulatory molecule or a portion thereof. Non-limiting examples of costimulatory molecules include CD28, 4-1BB, OX40, DAP10, and ICOS. In certain embodiments, the costimulatory molecule is CD28. In certain embodiments, the costimulatory molecule is 4-1BB.

In certain embodiments, the activating domain is included within one CAR, whereas the costimulatory component is provided by another CAR recognizing another antigen. In certain embodiments, the CARs include activating or stimulatory CARs, costimulatory CARs, both expressed on the same cell (see WO2014/055668). In certain embodiments, the cells include one or more stimulatory or activating CAR and/or a costimulatory CAR. In certain embodiments, the cells further include inhibitory CARs (“iCARs”, see Fedorov et al., Sci. Transl. Medicine (2013); 5:215) as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In certain embodiments, the intracellular signaling domain comprises a 4-1BB transmembrane and signaling domain linked to a CD3 (e.g., CD3-zeta) intracellular domain. In certain embodiments, the intracellular signaling domain comprises a chimeric CD28 and CD137 (4-1BB, TNFRSF9) co-stimulatory domains, linked to a CD3 zeta intracellular domain.

In certain embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In certain embodiments, the recombinant receptor further includes a marker and/or cells expressing the CAR or other antigen receptor further includes a surrogate marker, such as a cell surface marker, which may be used to confirm transduction or engineering of the cell to express the receptor. In certain embodiments, the marker includes all or part (e.g., truncated form) of CD34, a NGFR, or epidermal growth factor receptor, such as truncated version of such a cell surface receptor (e.g., tEGFR). In certain embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (EGFRt) that is, optionally, linked to a linker sequence, such as a T2A cleavable linker sequence.

In certain embodiments, the marker is a molecule, e.g., cell surface protein, not naturally found on T cells or not naturally found on the surface of T cells, or a portion thereof. In certain embodiments, the molecule is a non-self molecule, e.g., non-self protein, i.e., one that is not recognized as “self” by the immune system of the host into which the cells will be adoptively transferred.

In certain embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In certain embodiments, CARs are referred to as first, second, and/or third generation CARs. In certain embodiments, a first generation CAR is one that solely provides a CD3-chain induced signal upon antigen binding, e.g., does not comprise a costimulatory signaling region. In certain embodiments, a second-generation CARs is one that provides such a signal and costimulatory signal, such as a CAR including a costimulatory signaling region, e.g., an intracellular signaling domain or a portion thereof of a costimulatory molecule, such as CD28 or 4-1BB. In certain embodiments, a third generation CAR is one that includes multiple costimulatory regions, e.g., intracellular signaling domains of different costimulatory molecules (e.g., CD28 and 4-1BB).

In certain embodiments, the CAR includes an antibody, e.g., an antibody fragment, a transmembrane domain that is or includes a transmembrane portion of CD28 or a functional variant thereof, and an intracellular signaling domain comprising a signaling portion of CD28 or a functional variant thereof and a signaling portion of CD3 zeta or afunctional variant thereof. In certain embodiments, the CAR includes an antibody, e.g., an antibody fragment, a transmembrane domain that is or includes a transmembrane portion of CD28 or a functional variant thereof, and an intracellular signaling domain comprising a signaling portion of a 4-1BB or a functional variant thereof and a signaling portion of CD3 zeta or a functional variant thereof. In certain embodiments, the CAR includes an antibody, e.g., an antibody fragment, a transmembrane domain that is or includes a transmembrane portion of CD8 or a functional variant thereof, and an intracellular signaling domain comprising a signaling portion of a 4-1BB or a functional variant thereof and a signaling portion of CD3 zeta or a functional variant thereof. In certain embodiments, the receptor further includes a spacer comprising a portion of an Ig molecule, such as a human Ig molecule, such as an Ig hinge, e.g. an IgG4 hinge, such as a hinge-only spacer.

In certain embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In certain embodiments, the CAR includes an antibody or a fragment thereof, such as scFv, a spacer such as any of the Ig-hinge containing spacers, a CD28-derived transmembrane domain, a 4-1BB-derived intracellular signaling domain, and a CD3 zeta-derived signaling domain. In certain embodiments, the CAR includes an antibody or a fragment thereof, such as scFv, a spacer such as any of the Ig-hinge containing spacers, a CD8-derived transmembrane domain, a 4-1BB-derived intracellular signaling domain, and a CD3 zeta-derived signaling domain.

In certain embodiments, a nucleic acid molecule encoding such CAR construct further includes a sequence encoding a T2A ribosomal skip element and/or a tEGFR sequence, e.g., downstream of the sequence encoding the CAR. In certain embodiments, T cells expressing a recombinant receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374).

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, In certain embodiments, the cells express a CAR that specifically binds to an antigen expressed by a cell or tissue of the disease or condition or associated with the disease or condition.

### 7.3.2. TCRS

In certain embodiments, the recombinant receptors include recombinant T cell receptors (TCRs) and/or TCRs cloned from naturally occurring T cells. In certain embodiments, a high-affinity T cell clone for a target antigen (e.g., a cancer antigen) is identified, isolated from a patient, and introduced into the cells. In certain embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In certain embodiments, phage display is used to isolate TCRs against a target antigen (see, e.g., Varela-Rohena et al. (2008) Nat Med. 14:1390-1395 and Li (2005) Nat Biotechnol. 23:349-354.

In certain embodiments, after the T-cell clone is obtained, the TCR alpha and beta chains are isolated and cloned into a gene expression vector. In certain embodiments, the TCR alpha and beta genes are linked via a picornavirus 2A ribosomal skip peptide so that both chains are coexpression. In certain embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; an Hackett et al. (2010) Molecular Therapy: The Journal of the American Society of Gene Therapy. 18:674-683.

### 7.4. GENETICALLY ENGINEERED CELLS AND METHODS OF PRODUCING CELLS

In certain embodiments, the provided methods include administering to a subject having a disease or condition cells expressing a recombinant receptor. Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

### 7.4.1. VECTORS AND METHODS FOR GENETIC ENGINEERING

In certain embodiments, the recombinant nucleic acids are transferred into cells using recombinant infectious virus particles, such as, e.g., vectors derived from simian virus 40 (SV40), adenoviruses, adeno-associated virus (AAV). In certain embodiments, the recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In certain embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In certain embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In certain embodiments, the recombinant nucleic acids are transferred into the cells via electroporation (see, e.g., Chicaybam et al, (2013) PLoS ONE 8(3): e60298 and Van Tedeloo et al. (2000) Gene Therapy 7(16): 1431-1437). In certain embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In certain embodiments, the cells (e.g., T cells) may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/WIC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells (e.g., T cells) are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### 7.4.2. CELLS AND PREPARATION OF CELLS FOR GENETIC ENGINEERING

In certain embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In certain embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In certain embodiments, the cells are derived from the blood, bone marrow, lymph, or lymphoid organs, are cells of the immune system, such as cells of the innate or adaptive immunity. In certain embodiments, the cell is a cell of the lymphoid lineage or a cell of the myeloid lineage. In certain embodiments, the cell is a cell of the lymphoid lineage. In certain embodiments, the cell of the lymphoid lineage is selected from the group consisting of a T cell, a Natural Killer (NK) cell, a stem cell from which lymphoid cells may be differentiated. Stem cells can be multipotent and pluripotent stem cells. In certain embodiments, the stem cell is a pluripotent stem cell. In certain embodiments, the pluripotent stem cell is an embryoid stem cell or an induced pluripotent stem cell. The cells typically are primary cells, such as those isolated directly from a subject and/or isolated from a subject and frozen. In certain embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In certain embodiments, such as for off-the-shelf technologies, the cells are pluripotent and/or multipotent, such as stem cells, such as induced pluripotent stem cells (iPSCs). In certain embodiments, the methods include isolating cells from the subject, preparing, processing, culturing, and/or engineering them, and re-introducing them into the same subject, before or after cryopreservation.

In certain embodiments, the cells are T cells. Non-limiting examples of sub-types and subpopulations of T cells and/or of CD4⁺ and/or of CD8⁺ T cells include naïve T (TN) cells, Natural Killer T cells, effector T cells (T_{EFF}), memory T cells and sub-types thereof, such as stem cell memory T (T_{SCM}), central memory T (T_{CM}), effector memory T (T_{EM}), terminally differentiated effector memory T cells, tumor-infiltrating lymphocytes (TILs), immature T cells, mature T cells, helper T cells, cytotoxic T cells, mucosa-associated invariant T (MAIT) cells, regulatory T (Treg) cells, helper T cells (e.g., TH1 cells, TH2 cells, TH3 cells, TH17 cells, TH9 cells, TH22 cells, and follicular helper T cells), alpha/beta T cells, and delta/gamma T cells.

In certain embodiments, the cells are natural killer (NK) cells. In certain embodiments, the cells are monocytes or granulocytes, e.g., myeloid cells, macrophages, neutrophils, dendritic cells, mast cells, eosinophils, and/or basophils.

In certain embodiments, the cells are cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the cells include one or more nucleic acid molecules introduced via genetic engineering, and thereby express recombinant or genetically engineered products of such nucleic acids. In certain embodiments, the nucleic acid molecules are heterologous, i.e., normally not present in a cell or sample obtained from the cell, such as one obtained from another organism or cell, which for example, is not ordinarily found in the cell being engineered and/or an organism from which such cell is derived. In certain embodiments, the nucleic acid molecules are not naturally occurring, such as a nucleic acid molecule not found in nature, including one comprising chimeric combinations of nucleic acid molecules encoding various domains from multiple different cell types.

In certain embodiments, preparation of the engineered cells includes one or more culture and/or preparation steps. The cells for introduction of the nucleic acid molecule encoding the transgenic receptor such as the CAR, may be isolated from a sample, such as a biological sample, e.g., one obtained from or derived from a subject. In certain embodiments, the subject from which the cell is isolated is one having the disease or condition or in need of a cell therapy or to which cell therapy will be administered. In certain embodiments, the subject is a human in need of a particular therapeutic intervention, such as the adoptive cell therapy for which cells are being isolated, processed, and/or engineered.

Accordingly, the cells in certain embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain embodiments, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In certain embodiments, the cells are derived from cell lines, e.g., T cell lines. In certain embodiments, the cells are obtained from a xenogeneic source, for example, from mouse, rat, non-human primate, and pig.

In certain embodiments, isolation of the cells includes one or more preparation and/or non-affinity based cell separation steps. In certain embodiments, cells are washed, centrifuged, and/or incubated in the presence of one or more reagents, for example, to remove unwanted components, enrich for desired components, lyse or remove cells sensitive to particular reagents. In certain embodiments, cells are separated based on one or more property, such as density, adherent properties, size, sensitivity and/or resistance to particular components.

In certain embodiments, cells from the circulating blood of a subject are obtained, e.g., by apheresis or leukapheresis. The samples, in certain embodiments, comprise lymphocytes, including T cells, monocytes, granulocytes, B cells, other nucleated white blood cells, red blood cells, and/or platelets, and in certain embodiments includes cells other than red blood cells and platelets.

In certain embodiments, the blood cells collected from the subject are washed, e.g., to remove the plasma fraction and to place the cells in an appropriate buffer or media for subsequent processing steps. In certain embodiments, the cells are washed with phosphate buffered saline (PBS). In certain embodiments, the wash solution lacks calcium and/or magnesium and/or many or all divalent cations. In certain embodiments, a washing step is accomplished a semi-automated “flow-through” centrifuge (for example, the Cobe 2991 cell processor, Baxter) according to the manufacturer's instructions. In certain embodiments, a washing step is accomplished by tangential flow filtration (TFF) according to the manufacturer's instructions. In certain embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In certain embodiments, the methods include density-based cell separation methods, such as the preparation of white blood cells from peripheral blood by lysing the red blood cells and centrifugation through a Percoll or Ficoll gradient.

In certain embodiments, the isolation methods include separation of different cell types based on the expression or presence in the cell of one or more specific molecules, such as surface markers, e.g., surface proteins, intracellular markers, or nucleic acid. In certain embodiments, any known method for separation based on such markers may be used. In certain embodiments, the separation is affinity- or immunoaffinity-based separation. For example, the isolation in certain embodiments includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In certain embodiments, both fractions are retained for further use. In certain embodiments, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In certain embodiments, multiple rounds of separation steps are carried out, where the positively or negatively selected fraction from one step is subjected to another separation step, such as a subsequent positive or negative selection. In certain embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in certain embodiments, specific subpopulations of T cells, such as cells positive or expressing detection (e.g., high) levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using CD3/CD28 conjugated magnetic beads (e.g., DYNABEADS® M-450 CD3/CD28 T Cell Expander).

In certain embodiments, isolation is carried out by enrichment for a particular cell population by positive selection, or depletion of a particular cell population, by negative selection. In certain embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In certain embodiments, T cells are separated from a PBMC sample by negative selection of markers expressed on non-T cells, such as B cells, monocytes, or other white blood cells, such as CD14. In certain embodiments, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In certain embodiments, CD8⁺ cells are further enriched for or depleted of naive, central memory, effector memory, and/or central memory stem cells, such as by positive or negative selection based on surface antigens associated with the respective subpopulation. In certain embodiments, enrichment for central memory T (T_{CM}) cells is carried out to increase efficacy, such as to improve long-term survival, expansion, and/or engraftment following administration, which in certain embodiments is particularly robust in such sub-populations. See Terakura et al. (2012) Blood. 1:72-82; Wang et al. (2012) J Immunother. 35(9):689-701. In certain embodiments, combining T_{CM}-enriched CD8⁺ T cells and CD4⁺ T cells further enhances efficacy.

In certain embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In certain embodiments, the enrichment for central memory T (T_{CM}) cells is based on positive or high surface expression of CD45RO, CD62L, CCR7, CD28, CD3, and/or CD 127; in certain embodiments, it is based on negative selection for cells expressing or highly expressing CD45RA and/or granzyme B. In certain embodiments, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in certain embodiments are carried out simultaneously and in other aspects are carried out sequentially, in either order. In certain embodiments, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In certain embodiments, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In certain embodiments, naive CD4⁺ T lymphocytes are CD45RO⁻, CD45RA⁺, CD62L⁺, CD4⁺ T cells. In certain embodiments, central memory CD4⁺ cells are CD62L⁺ and CD45RO⁺. In certain embodiments, effector CD4⁺ cells are CD62L⁻ and CD45RO⁻.

In certain embodiments, to enrich for CD4⁺ cells by negative selection, a monoclonal antibody cocktail typically includes antibodies to CD14, CD20, CD11b, CD16, HLA-DR, and CD8. In certain embodiments, the antibody or binding partner is bound to a solid support or matrix, such as a magnetic bead or paramagnetic bead, to allow for separation of cells for positive and/or negative selection. For example, in certain embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In certain embodiments, the preparation methods include steps for freezing, e.g., cryopreserving, the cells, either before or after isolation, incubation, and/or engineering. In certain embodiments, the freeze and subsequent thaw step removes granulocytes and, to some extent, monocytes in the cell population. In certain embodiments, the cells are suspended in a freezing solution, e.g., following a washing step to remove plasma and platelets. Any of a variety of known freezing solutions and parameters in certain embodiments may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In certain embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In certain embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In certain embodiments, the stimulating conditions or agents include one or more agent, e.g., ligand, which is capable of activating an intracellular signaling domain of a TCR complex. In certain embodiments, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In certain embodiments, the stimulating agents include IL-2 and/or IL-15, for example, an IL-2 concentration of at least about 10 units/mL.

In certain embodiments, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In certain embodiments, the T cells are expanded by adding to a culture-initiating composition feeder cells, such as non-dividing peripheral blood mononuclear cells (PBMC), (e.g., such that the resulting population of cells includes at least about 5, 10, 20, or 40 or more PBMC feeder cells for each T lymphocyte in the initial population to be expanded); and incubating the culture (e.g. for a time sufficient to expand the numbers of T cells). In certain embodiments, the non-dividing feeder cells can comprise gamma-irradiated PBMC feeder cells. In certain embodiments, the PBMC are irradiated with gamma rays in the range of about 3000 to 3600 rads to prevent cell division. In certain embodiments, the feeder cells are added to culture medium prior to the addition of the populations of T cells.

In certain embodiments, the stimulating conditions include temperature suitable for the growth of human T lymphocytes, for example, at least about 25 degrees Celsius, generally at least about 30 degrees, and generally at about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in certain embodiments is provided in any suitable amount, such as a ratio of LCL feeder cells to initial T lymphocytes of at least about 10:1.

In embodiments, antigen-specific T cells, such as antigen-specific CD4⁺ and/or CD8⁺ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### 7.5. COMPOSITIONS AND FORMULATIONS OF GENETICALLY ENGINEERED CELLS

In certain embodiments, the cells engineered with a recombinant receptor, e.g. CAR or TCR, is provided as a composition or formulation, such as a pharmaceutical composition or formulation. Such compositions can be used in accord with the provided methods, such as in the prevention or treatment of diseases, conditions, and disorders, or in detection, diagnostic, and prognostic methods. The term “pharmaceutical formulation” refers to a preparation which is in such form as to permit the biological activity of an active ingredient contained therein to be effective, and which includes no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In certain embodiments, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In certain embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in certain embodiments are included in the compositions. Suitable buffering agents include, for example, citric acid, sodium citrate, phosphoric acid, potassium phosphate, and various other acids and salts. In certain embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in certain embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In certain embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the agent or host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

The pharmaceutical composition in certain embodiments can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

In certain embodiments, the pharmaceutical composition includes agents or cells in amounts effective to treat or prevent the disease or condition, such as a therapeutically effective or prophylactically effective amount. In certain embodiments, therapeutic or prophylactic efficacy is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In certain embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in certain embodiments, the cells are derived from a subject, e.g., patient, in need of a treatment and the cells, and following isolation and processing are administered to the same subject.

In certain embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In certain embodiments, the first and second subjects are genetically identical. In certain embodiments, the first and second subjects are genetically similar. In certain embodiments, the second subject expresses the same HLA class or supertype as the first subject.

The agents or cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In certain embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In certain embodiments, a given dose is administered by a single bolus administration of the cells or agent. In certain embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. In certain embodiments, the compositions are suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or heterologous. For example, cells or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified cell or an agent that treats or ameliorates symptoms of neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In certain embodiments, the agent or cell populations are administered parenterally. The term “parenteral,” as used herein, includes intravenous, intramuscular, subcutaneous, rectal, vaginal, and intraperitoneal administration. In certain embodiments, the agent or cell populations are administered to a subject using peripheral systemic delivery by intravenous, intraperitoneal, or subcutaneous injection.

In certain embodiments, compositions are provided as sterile liquid preparations, e.g., isotonic aqueous solutions, suspensions, emulsions, dispersions, or viscous compositions, which may in certain embodiments be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in certain embodiments be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### 7.6. DOSING OF THE GENETICALLY ENGINEERED CELLS

In certain embodiments, the size or timing of the doses of the genetically engineered cells is determined as a function of the particular disease or condition in the subject. It is within the level of a skilled artisan to empirically determine the size or timing of the doses for a particular disease in view of the provided description. For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. In certain embodiments, the compositions and cells are suitably administered to the subject at one time or over a series of treatments.

In the context of adoptive cell therapy, administration of a given “dose” referring to the cells encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the first or consecutive dose is a single or continuous administration of the specified number of cells, given or initiated at a single point in time. In some contexts, however, the first or consecutive dose is administered in multiple injections or infusions over a period of no more than three days, such as once a day for three days or for two days or by multiple infusions over a single day period.

Thus, in certain embodiments, the cells of the first dose are administered in a single pharmaceutical composition. In certain embodiments, the cells of the consecutive dose are administered in a single pharmaceutical composition.

In certain embodiments, the cells of the first dose are administered in a plurality of compositions, collectively containing the cells of the first dose. In certain embodiments, the cells of the consecutive dose are administered in a plurality of compositions, collectively containing the cells of the consecutive dose. In certain embodiments, additional consecutive doses may be administered in a plurality of compositions over a period of no more than 3 days.

The quantity of cells to be administered will vary for the subject being treated. In certain embodiments, between about 1×10⁴ and about 1×10¹⁰, between about 1×10⁵ and about 1×10⁹, or between about 1×10⁶ and about 1×10⁸ of the genetically engineered cells (recombinant receptor-expressing cells) are administered to a subject (e.g., a human subject). More effective cells may be administered in even smaller numbers. In certain embodiments, at least about 1×10⁶, at least about 1×10⁷, at least about 1×10⁸ (e.g., about 2×10⁸, about 3×10⁸, about 4×10⁸, or about 5×10⁸) of the genetically engineered cells (recombinant receptor-expressing cells) are administered to a subject (e.g., a human subject). The precise determination of what would be considered an effective dose may be based on factors individual to each subject, including their size, age, sex, weight, and condition of the particular subject. Dosages can be readily ascertained by those skilled in the art from this disclosure and the knowledge in the art.

In certain embodiments, the numbers and/or concentrations of cells refer to the number of recombinant receptor (e.g., CAR)-expressing cells. In certain embodiments, the numbers and/or concentrations of cells refer to the number or concentration of all cells, T cells, or peripheral blood mononuclear cells (PBMCs) administered.

In certain embodiments, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In certain embodiments, the size of the dose is determined by the burden of the disease or condition in the subject. For example, in certain embodiments, the number of cells administered is determined based on the tumor burden that is present in the subject immediately prior to administration of the dose of cells. In certain embodiments, the size of the dose is inversely correlated with disease burden. In certain embodiments, as in the context of a large disease burden, the subject is administered a low number of cells.

In reference to cell numbers, in certain embodiments, such values refer to numbers of recombinant receptor-expressing (e.g. CAR-expressing) cells; in other embodiments, they refer to number of T cells or PBMCs or total cells administered.

In certain embodiments, one or more further consecutive doses can be administered. In certain embodiments, the number of cells administered in the consecutive dose is the same as or similar to the number of cells administered in the first dose in any of the embodiments herein. In certain embodiments, the particular dosage regimen is chosen to reduce or minimize toxicity in the subject upon administration of the cells expressing the recombinant receptor, such as described in International Patent Application Publication No. WO2016/064929, which is incorporated by reference herein.

In certain embodiments, if desired or need in a particular disease or context, the provided methods involve a consecutive dose of cells administered at an increased number, and hence at a higher dose, than the first dose of cells. In certain embodiments, methods of first administering a low dose of recombinant receptor-expressing (e.g., CAR-expressing) cells can reduce the disease burden in subjects, such as from morphological disease status to minimal disease, so that subsequent administration of a higher dose of recombinant receptor-expressing (e.g., CAR-expressing) cells in subjects is less likely to cause toxic outcomes in a majority of subjects treated.

In certain embodiments, a subject can be assessed for tumor burden after administration of the first dose and prior to administration of the consecutive dose to confirm that tumor burden has been reduced compared to tumor burden present prior to treatment with the first dose. In certain embodiments, if assessment of the subject indicates tumor burden is reduced and/or that the subject exhibits non-morphological disease, for example molecularly detectable disease and/or minimal disease, the provided methods include administering a consecutive dose of recombinant receptor-expressing (e.g., CAR-expressing) cells that is the same or less than the first or initial dose. In certain embodiments, if assessment of the subject indicates tumor burden is not reduced and/or the subject exhibits morphological disease, the provided methods include administering a consecutive dose of recombinant receptor-expressing (e.g., CAR-expressing) cells that is higher than the first or initial dose.

In certain embodiments, a consecutive dose of recombinant receptor-expressing (e.g., CAR-expressing) cells is administered to the subject at a time after administration of the first or initial dose of cells in which it is likely that tumor burden of the subject has been reduced by the first dose. In certain embodiments, it is not necessary that the tumor burden actually be reduced in all subjects prior to administration of the consecutive dose, but that tumor burden is reduced on average in subjects treated, such as based on clinical data, in which a majority of subjects treated with such a first dose exhibit a reduced tumor burden, such as at least about 50%, about 60%, about 70%, about 80%, about 90%, about 95% or more of subjects treated with the first or initial dose exhibit a reduced tumor burden. Generally at a point in time after disease burden has been reduced by the first dose or is likely to have been reduced by the first dose, a consecutive dose is administered to the subject, thereby further reducing and/or eliminating disease or a symptom or outcome thereof or preventing expansion or progression thereof. The context of reduced disease burden at the time of the consecutive administration in certain embodiments reduces the likelihood of exhaustion of the transferred cells, thereby improving efficacy. The consecutive dose may be the same, lower, or a higher dose as compared with the first dose. In certain embodiments, multiple consecutive doses are administered after a first dose.

In certain embodiments, the consecutive dose of recombinant receptor-expressing (e.g., CAR-expressing) cells is administered at a dose that is higher than the first dose so that an increased number of recombinant receptor-expressing (e.g., CAR-expressing) cells is administered to the subject by the consecutive dose. In certain embodiments, a higher dose of recombinant receptor-expressing (e.g., CAR-expressing) cells is one that can promote an increased response or efficacy, such as improved or greater reduction in tumor burden and/or an improved or greater overall survival time of the subject compared to that achieved by administration of a lower dose or number of cells. In certain embodiments, because administration of the first dose of cells can reduce tumor burden in the subject, administration of the consecutive dose at a higher number of cells can avoid or minimize CRS and/or neurotoxicity in the subject after administration of the consecutive dose that can otherwise occur in subjects with morphological disease.

In certain embodiments, the consecutive dose is larger than the first dose. For example, in certain embodiments, the consecutive dose includes more than about 1×10⁶ cells (e.g., about or at least about 2×10⁶, about 3×10⁶, about 5×10⁶, about 1×10⁷, about 1×10⁸, or about 1×10⁹) of the recombinant receptor (e.g. CAR)-expressing cells. In certain embodiments, the amount or size of the consecutive dose is sufficient to reduce disease burden or an indicator thereof, and/or one or more symptoms of the disease or condition. In certain embodiments, the dose is of a size effective to improve survival of the subject, for example, to induce survival, relapse-free survival, or event-free survival of the subject for at least 6 months, or at least about 1, about 2, about 3, about 4, or about 5 years. In certain embodiments, the number of recombinant receptor (e.g. CAR)-expressing cells (e.g., CAR-expressing T cells) administered and/or number of such cells administered per body weight of the subject in the consecutive dose is at least about 2-fold, about 3-fold, about 5-fold, about 10-fold greater than the number administered in the first dose. In certain embodiments, disease burden, tumor size, tumor volume, tumor mass, and/or tumor load or bulk is reduced following the consecutive dose by at least about 50%, about 60%, about 70%, about 80%, or about 90% or more compared to that immediately prior to the administration of the first dose or of the consecutive dose.

In certain embodiments, the number of cells administered in the consecutive dose is lower than the number of cells administered in the first dose.

In certain embodiments, multiple consecutive doses are administered following the first dose, such that an additional dose or doses are administered following administration of the consecutive dose. In certain embodiments, the number of cells administered to the subject in the additional dose or doses (i.e., the third, fourth, fifth, and so forth) is the same as or similar to the first dose and/or consecutive dose. In certain embodiments, the additional dose or doses are larger than prior doses.

In certain embodiments, the timing of the consecutive dose(s) in relation to the first and/or one another is designed to reduce the risk of unwanted toxic outcomes and promote maximum efficacy. In certain embodiments, the consecutive dose, such as the same, lower or higher consecutive dose, is administered at a time at which disease burden remains reduced in the subject or reduced in subjects on average, such as based on clinical data, but at which the risk of CRS and/or neurotoxicity remain low. In certain embodiments, a consecutive dose is generally given at a point in time relative to the first or previous dose at which the risk of a toxic outcome or symptom or biochemical indicator thereof—such as CRS or neurotoxicity, macrophage activation syndrome, or tumor lysis syndrome—is at or below an acceptable level. For example, the consecutive dose may be administered after a toxic outcome has peaked and is declining or has declined below an acceptable level following the initial dose. In certain embodiments, the appropriate timing is determined by monitoring and/or assessing the presence of one or more symptoms or outcomes associated with the toxic event and delivering the consecutive dose after determining that the symptom or outcome is at or below an acceptable level.

Once the cells are administered to the subject (e.g., human), the biological activity of the engineered cells can be measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In certain embodiments, the biological activity is measured by assessing clinical outcome, such as reduction in tumor burden or load. In certain embodiments, toxic outcomes, persistence and/or expansion of the cells, and/or presence or absence of a host immune response, are assessed.

### 7.7. RADIATION THERAPY

Radiation therapies which are suitable for use in the combination treatments described herein, include, but not limited to, external beam radiation, a radiopharmaceutical agent which comprises a radiation-emitting radioisotope, internal radiation therapy, radionuclide therapy, and radiation surgery.

In certain embodiments, the presently disclosed combination methods include use of external beam radiation therapy. External beam radiation therapy uses a radiation (ionizing radiation) source that is external to the subject (e.g., at the region of the subject that contains the lesion), typically either a radioisotope, such as ⁶⁰Co, ¹³⁷Cs, or a high energy x-ray source, such as a linear accelerator. In certain embodiments, external beam radiation therapy comprises orthovoltage (i.e., superficial) beams of radiation to treat and/or disrupt a lesion present on the skin. In certain embodiments, external beam radiation therapy comprises megavoltage, e.g., deep, beams of radiation are used to treat internal lesions, e.g., lesions of the bladder, bowel, prostate, lung, or brain. In certain embodiments, external beam radiation therapy comprises delivering X rays, gamma rays, electron beams, proton beams, or beams of ionized nuclei to the lesion. In certain embodiments, the external beam radiation therapy is performed with a linear accelerator, a collimator, a cobalt machine, a superficial radiation therapy (SRT) machine, Orthovoltage X ray machine.

The external source produces a collimated beam directed into the patient to the lesion (e.g., tumor) site. The adverse effect of irradiating of healthy tissue can be reduced, while maintaining a given dose of radiation in the tumorous tissue, by projecting the external radiation beam into the patient at a variety of “gantry” angles with the beams converging on the lesion (e.g., tumor) site. The particular volume elements of healthy tissue, along the path of the radiation beam, change, reducing the total dose to each such element of healthy tissue during the entire treatment. The irradiation of healthy tissue also can be reduced by tightly collimating the radiation beam to the general cross section of the tumor taken perpendicular to the axis of the radiation beam. Numerous systems exist for producing such a circumferential collimation, some of which use multiple sliding shutters which, piecewise, can generate a radio-opaque mask of arbitrary outline.

In certain embodiments, the presently disclosed combination methods include administering to the subject a radiopharmaceutical agent. A “radiopharmaceutical agent”, as defined herein, refers to a pharmaceutical agent which includes at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, includes a radioisotope (RI) that serves as the radiation source.

As used herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

As used herein, a “metallic radioisotope” is any suitable metallic radioisotope useful in a therapeutic or diagnostic procedure in vivo or in vitro. Suitable metallic radioisotopes include, but are not limited to: Actinium-225, Antimony-124, Antimony-125, Arsenic-74, Barium-103, Barium-140, Beryllium-7, Bismuth-206, Bismuth-207, Bismuth 212, Bismuth 213, Cadmium-109, Cadmium-115m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-60, Copper-62, Copper-64, Copper-67, Erbium-169, Europium-152, Gallium-64, Gallium-67, Gallium-68, Gadolinium 153, Gadolinium-157 Gold-195, Gold-199, Hafnium-175, Hafnium-175-181, Holmium-166, Indium-110, Indium-111, Iridium-192, Iron 55, Iron-59, Krypton 85, Lead-203, Lead-210, Lutetium-177, Manganese-54, Mercury-197, Mercury 203, Molybdenum-99, Neodymium-147, Neptunium-237, Nickel-63, Niobium 95, Osmium-185+191, Palladium-103, Palladium-109, Platinum-195m, Praseodymium-143, Promethium-147, Promethium-149, Protactinium-233, Radium-226, Rhenium-186, Rhenium-188, Rubidium-86, Ruthenium-97, Ruthenium-103, Ruthenium-105, Ruthenium-106, Samarium-153, Scandium-44, Scandium-46, Scandium-47, Selenium-75, Silver-110m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65, Zirconium-89, Zirconium-95, and Dysprosium-165.

As used herein, a “non-metallic radioisotope” is any suitable nonmetallic radioisotope (non-metallic radioisotope) useful in a therapeutic or diagnostic procedure in vivo or in vitro. Suitable non-metallic radioisotopes include, but are not limited to: Iodine-131, Iodine-125, Iodine-123, Phosphorus-32, Astatine-211, Fluorine-18, Carbon-11, Oxygen-15, Bromine-76, and Nitrogen-13.

One of ordinary skill in the art may select a specific biomolecule for use in targeting a particular neoplasm for radionuclide therapy based upon the cell-surface molecules present on that neoplasm.

Identifying the most appropriate isotope for radiotherapy requires weighing a variety of factors. These include tumor uptake and retention, blood clearance, rate of radiation delivery, half-life and specific activity of the radioisotope, and the feasibility of large-scale production of the radioisotope in an economical fashion. The key point for a therapeutic radiopharmaceutical is to deliver the requisite amount of radiation dose to the tumor cells and to achieve a cytotoxic or tumoricidal effect while not causing unmanageable side-effects.

In certain embodiments, the physical half-life of the therapeutic radioisotope is similar to the biological half-life of the radiopharmaceutical at the lesion (e.g., tumor) site. For example, if the half-life of the radioisotope is too short, much of the decay may have occurred before the radiopharmaceutical has reached maximum target/background ratio. On the other hand, too long a half-life may cause unnecessary radiation dose to normal tissues. Ideally, the radioisotope should have a long enough half-life to attain a minimum dose rate and to irradiate all the cells during the most radiation sensitive phases of the cell cycle. In addition, the half-life of a radioisotope has to be long enough to allow adequate time for manufacturing, release, and transportation.

Other practical considerations in selecting a radioisotope for a given application in tumor therapy are availability and quality. The purity has to be sufficient and reproducible, as trace amounts of impurities can affect the radiolabeling and radiochemical purity of the radiopharmaceutical.

The target receptor sites in tumors are typically limited in number. As such, in certain embodiments, the radioisotope have high specific activity. The specific activity depends primarily on the production method. Trace metal contaminants must be minimized as they often compete with the radioisotope for the chelator and their metal complexes compete for receptor binding with the radiolabeled chelated agent.

In certain embodiments, the radiation treatment used in the combination methods of the present disclosure is a combination of external beam radiation and a radioisotope, such as a radiopharmaceutical agent.

In certain embodiments, the presently disclosed combination methods include use of internal radiation therapy, e.g., brachytherapy. In certain embodiments, the brachytherapy comprises applying sources of radiation at or near the area of the lesion. In certain embodiments, the brachytherapy comprises interstitial radiation wherein the radiation source is contained in small pellets, seeds, wires, tubes, and/or containers and is placed directly into or next to the lesion. In certain embodiments, the brachytherapy comprises intracavitary radiation, wherein a container of radioactive material is placed in a cavity of the body, e.g., chest cavity or large intestine. In certain embodiments, ultrasounds, X-rays, and/or CT scans are used to assist with the placement of the radioactive source.

In certain embodiments, the presently disclosed combination methods include use of permanent brachytherapy, which comprises placing small containers, e.g., containers approximately the size of a grain of rice, into a lesion. In certain embodiments, containers give off radiation for a time period of several weeks or months, and are left in place after the radiation is used up.

In certain embodiments, the presently disclosed combination methods include use of temporary brachytherapy which comprises placing cylinders, hollow needles, tubes (catheters), and/or fluid-filled balloons into the area to be treated that are then removed after treatment. In certain embodiments, radioactive materials are placed in these containers for a short time and then removed. In certain embodiments, the temporary brachytherapy can be high-dose rate (HDR) brachytherapy, wherein the radiation source is put into place for a few minutes at a time at or near the lesion, and then is removed. This process may be repeated twice a day for up to a week, or once a week for a few weeks. In certain embodiments, the temporary brachytherapy is low dose rate (LDR) brachytherapy, wherein the radiation source stays in place for up to 7 days before it is removed.

The type of radiation that is suitable for use in the combination methods disclosed herein can vary. For example, radiation can be electromagnetic or particulate in nature. Electromagnetic radiation useful in the practice of the presently disclosed combination methods includes, but is not limited to, x-rays and gamma rays. Particulate radiation useful in the practice of the presently disclosed combination methods includes, but is not limited to, electron beams (beta particles), protons beams, neutron beams, alpha particles, and negative pi mesons.

The radiation can be delivered using conventional radiological treatment apparatus and methods, and by intraoperative and stereotactic methods. Additional discussion regarding radiation treatments suitable for use in the practice of the presently disclosed combination methods can be found throughout Steven A. Leibel et al., Textbook of Radiation Oncology (1998); (publ. W. B. Saunders Company), and particularly in Chapters 13 and 14. Radiation can also be delivered by other methods such as targeted delivery, for example by radioactive “seeds,” or by systemic delivery of targeted radioactive conjugates. J.; Padawer et al., Combined Treatment with Radioestradiol lucanthone in Mouse C3HBA Mammary Adenocarcinoma and with Estradiol lucanthone in an Estrogen Bioassay, Int. J. Radiat. Oncol. Biol. Phys. 7: 347-357 (1981).

For tumor therapy, both α and β-particle emitters have been investigated. Alpha particles are particularly good cytotoxic agents because they dissipate a large amount of energy within one or two cell diameters. The (β-particle emitters have relatively long penetration range (2-12 mm in the tissue) depending on the energy level. The long-range penetration is particularly important for solid tumors that have heterogeneous blood flow and/or receptor expression. The (β-particle emitters yield a more homogeneous dose distribution even when they are heterogeneously distributed within the target tissue.

### 7.8. ADMINISTRATION OF RADIATION THERAPY

The combination methods of the present disclosure include administering to the subject an effective dose of radiation. The radiation can be provided by targeted delivery. The radiation can also be provided by systemic delivery (e.g., systemic delivery of targeted radioactive conjugates, for example a radiolabeled antibody).

### 7.8.1. ADMINISTRATION OF EXTERNAL BEAM RADIATION

For administration of external beam radiation, the amount can be at least about 1 Gray (Gy) fractions at least once every other day to a treatment volume at a lesion site. In certain embodiments, the radiation is administered in at least about 2 Gray (Gy) fractions at least once per day (daily) to a treatment volume at a lesion site. In certain embodiments, the radiation is administered in at least about 4 Gray (Gy) fractions at least once per day (daily) to a treatment volume at a lesion site. In certain embodiments, the radiation is administered in at least about 2 Gray (Gy) fractions at least once per day to a treatment volume for five consecutive days per week at a lesion site. In certain embodiments, the radiation is administered in at least about 4 Gray (Gy) fractions at least once per day to a treatment volume for five days per week at a lesion site. In certain embodiments, the radiation is administered in about 4 Gray (Gy) fractions daily for five days at a lesion site. In certain embodiments, the radiation is administered in at least about 4 Gray (Gy) fractions once per day for five consecutive days at a lesion site. In certain embodiments, radiation is administered in 10 Gy fractions every other day, three times per week to a treatment volume at a lesion site.

In certain embodiments, a total of at least between about 5 Gy and about 40 Gy (e.g., between about 5 Gy and about 30 Gy, between about 10 Gy and about 30 Gy, between about 5 Gy and about 20 Gy, between about 10 Gy and about 20 Gy, or between about 10 Gy and about 30 Gy) is administered to a lesion site of a subject in need thereof. In certain embodiments, a total of at least about 10 Gy is administered to a lesion site of a subject in need thereof. In certain embodiments, a total of at least about 20 Gy is administered to a lesion site of a subject in need thereof. In certain embodiments, at least about 30 Gy is administered to a lesion site of a subject in need thereof. In certain embodiments, at least about 40 Gy is administered to a lesion site of a subject in need thereof. In certain embodiments, a total of about 20 Gy is administered to a lesion site of a subject in need thereof.

Typically, the subject receives external beam therapy four or five times a week. An entire course of treatment usually lasts from one to seven weeks depending on the type of cancer and the goal of treatment. For example, a subject can receive a dose of 2 Gy/day over 30 days.

### 7.8.2. ADMINISTRATION OF RADIOPHARMACEUTICAL AGENT

There are a number of methods for administration of a radiopharmaceutical agent. For example, the radiopharmaceutical agent can be administered by targeted delivery or by systemic delivery of targeted radioactive conjugates, such as a radiolabeled antibody, a radiolabeled peptide and a liposome delivery system.

In certain embodiments of targeted delivery, the radiolabelled pharmaceutical agent can be a radiolabelled antibody. See, for example, Ballangrud A. M., et al. Cancer Res., 2001; 61: 2008-2014 and Goldenber, D. M. J. Nucl. Med., 2002; 43 (5): 693-713, the contents of which are incorporated by reference herein. In certain embodiments of targeted delivery, the radiopharmaceutical agent can be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. See, for example, Emfietzoglou D, Kostarelos K, Sgouros G. An analytical dosimetry study for the use of radionuclide-liposome conjugates in internal radiotherapy. J Nucl Med 2001; 42: 499-504, the contents of which are incorporated by reference herein.

In certain embodiments of targeted delivery, the radiolablled pharmaceutical agent can be a radiolabeled peptide. See, for example, Weiner R E, Thakur M L. Radiolabeled peptides in the diagnosis and therapy of oncological diseases. Appl Radiat Isot 2002 November; 57 (5): 749-63, the contents of which are incorporated by reference herein.

In addition to targeted delivery, Bracytherapy can be used to deliver the radiopharmaceutical agent to the target site. Brachytherapy is a technique that puts the radiation sources as close as possible to the lesion (e.g., tumor) site. Often the source is inserted directly into the lesion (e.g., tumor). The radioactive sources can be in the form of wires, seeds or rods. Generally, cesium, iridium or iodine are used.

The amount of radiation necessary can be determined by one of skill in the art based on known doses for a particular type of the disease or disorder (e.g., cancer). In certain embodiments, the radiation can be administered in amount effective to cause the arrest or regression of the cancer of, when the radiation is administered with the genetically engineered cells to provide the unexpected synergistic effect, e.g., a synergistic abscopal-like response. HDAC inhibitor.

### 7.9. REDUCTION IN DISEASE BURDEN, EFFICACY AND SURVIVAL

The administration in accord with the presently disclosed methods generally reduces or prevents the expansion or burden of the disease or condition in the subject. For example, where the disease or condition is a tumor, the methods generally reduce tumor size, bulk, metastasis, percentage of blasts in the bone marrow or molecularly detectable cancer and/or improve prognosis or survival or other symptom associated with tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood or bone marrow in the context of certain hematological malignancies. In certain embodiments, disease burden includes, the mass of a tumor, the number or extent of metastases and/or the percentage of blast cells present in the bone marrow.

In certain embodiments, the subject suffers from blood cancer. In certain embodiments, the subject suffers from multiple myeloma (“MM”). The extent of disease burden can be determined by assessment of residual MM in blood or bone marrow, e.g., based on the International Myeloma Working Group (IMWG) criteria for diagnosis for MM. In certain embodiments, the subject exhibits morphologic disease if a monoclonal paraprotein (M-spike) is detected (e.g., by serum protein electrophoresis (SPEP)). In certain embodiments, the subject exhibits morphologic disease if bone marrow infiltration by monoclonal malignant plasma cells (PC) is about 5% or greater. In certain embodiments, the subject exhibits morphologic disease if bone-based and/or extra-osseous MM lesions are detected. In certain embodiments, the subject exhibits complete or clinical remission if M-spike is undetectable, bone marrow infiltration by monoclonal malignant plasma cells (PC) is less than about 5%, and/or absence of bone-based and/or extra-osseous MM lesions.

In certain embodiments, the disease or condition persists following administration of the first dose and/or administration of the first dose is not sufficient to eradicate the disease or condition in the subject.

In certain embodiments, administration of the consecutive dose reduces disease burden as compared to disease burden at a time immediately prior to the first dose, or at a time immediately prior to the consecutive dose. In certain embodiments, for example in the context of relapse, administration of the consecutive dose effects a reduction in disease burden as compared to the peak level of disease burden following administration of the first dose.

In certain embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative dosing regimen, such as one including administrating genetically engineered cells without radiation therapy.

In certain embodiments, the burden of a disease or condition in the subject is detected, assessed, or measured. Disease burden may be detected in certain embodiments by detecting the total number of disease or disease-associated cells, e.g., tumor cells, in the subject, or in an organ, tissue, or bodily fluid of the subject, such as blood or serum. In certain embodiments, disease burden, e.g. tumor burden, is assessed by measuring the mass of a solid tumor and/or the number or extent of metastases. In certain embodiments, survival of the subject, survival within a certain time period, extent of survival, presence or duration of event-free or symptom-free survival, or relapse-free survival, is assessed. In certain embodiments, any symptom of the disease or condition is assessed. In certain embodiments, the measure of disease or condition burden is specified.

In certain embodiments, the event-free survival rate or overall survival rate of the subject is improved by the methods, as compared with other methods. For example, in certain embodiments, event-free survival rate or probability for subjects treated by the methods at about five weeks, about six weeks, about seven weeks, about eight weeks, about three months, about four months, about five months, about six months following the first dose is greater than about 40%, greater than about 50%, greater than about 60%, greater than about 70%, greater than about 80%, greater than about 90%, or greater than about 95%. In certain embodiments, overall survival rate is greater than about 40%, greater than about 50%, greater than about 60%, greater than about 70%, greater than about 80%, greater than about 90%, or greater than about 95%. In certain embodiments, the subject treated with the methods exhibits event-free survival, relapse-free survival, or survival to at least about five weeks, about six weeks, about seven weeks, about eight weeks, about three months, about four months, about five months, about six months, at least about 1 year, about 2 years, about 3 years, about 4 years, about 5 years, about 6 years, about 7 years, about 8 years, about 9 years, or about 10 years. In certain embodiments, the time to progression is improved, such as a time to progression of greater than about 6 months, at least about 1 year, about 2 years, about 3 years, about 4 years, about 5 years, about 6 years, about 7 years, about 8 years, about 9 years, or about 10 years following the first dose.

In certain embodiments, following treatment by the method, the probability of relapse is reduced as compared to other methods. For example, in certain embodiments, the probability of relapse at about five weeks, about six weeks, about seven weeks, about eight weeks, about three months, about four months, about five months, about six months following the first dose is less than about 80%, less than about 70%, less than about 60%, less than about 50%, less than about 40%, less than about 30%, less than about 20%, or less than about 10% as compared to other methods, e.g., one including administrating genetically engineered cells without radiation therapy.

### 7.10. KITS

Also provided are articles of manufacture, such as kits, which contain one or more cell doses according to any of the provided embodiments, such as in any of the formulations or compositions described (which may be present in one or more containers, such as bags or vials, with cells in sufficient numbers or concentration for administration of an individual dose or a portion thereof), and instructions and/or packaging materials or literature with instructions for the administration in accordance with a method as provided herein. In certain embodiments, the instructions indicate the caution or contraindication of any of the embodiments described.

### 8. EXAMPLES

The following examples are included for illustrative purposes only and are not intended to limit the scope of the presently disclosed subject matter.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

### Example 1—BCMA-Targeted CAR T-Cell Therapy Plus Radiation Therapy for the Treatment of Refractory Myeloma Reveals Potential Synergy

### Summary:

This Example presents a case of a patient with multiply-relapsed, refractory myeloma whose clinical course showed evidence of a synergistic abscopal-like response to CAR T-cell therapy and localized radiation therapy (XRT). Shortly after receiving B-cell maturation antigen (BCMA)—targeted CAR T-cell therapy, the patient required urgent high-dose steroids and XRT for spinal cord compression. Despite the steroids, the patient had a durable systemic response that could not be attributed to XRT alone. Post-XRT findings included a second wave of fever and increased CRP and IL6, beginning 21 days post-CAR T cells, which is late for cytokine release syndrome (CRS) from CAR T-cell therapy alone on this trial. Given this response, which resembled CRS, immediately following XRT, changes in the patient's T-cell receptor (TCR) repertoire were investigated over 10 serial time points. Comparing T-cell diversity via Morisita's Overlap Indices (C_{D}), it was discovered that, although the diversity was initially stable after CAR T-cell therapy compared with baseline (C_{D}=0.89-0.97, baseline vs 4 time points post-CAR T cells), T-cell diversity changed after the conclusion of XRT, with >30% newly expanded TCRs (C_{D}=0.56-0.69, baseline vs 4 time points after XRT). These findings suggest potential synergy between radiation and CAR T-cell therapies resulting in an abscopal-like response.

### Materials and Methods

Clinical protocol: The patient was treated on a trial evaluating autologous BCMA-targeted CAR T cells (Trial registration ID: NCT03070327; trial conducted in accordance with US Common Rule; informed written consent obtained from patients). Gene modification was accomplished via a retrovirus encoding a CAR that consists of a human-derived anti-BCMA scFv, a CD8 transmembrane domain, the signaling domains of 4-1BB and CD3ζ fused to a “self-cleaving” P2A element, and a separate gene encoding a surrogate transduction marker (Smith et al., Mol Ther. (2018 Jun. 6); 26(6):1447-1456). Under the trial protocol, patients underwent eukopheresis, and peripheral blood mononuclear cells (PBMCs) were frozen. At the appropriate time, PBMCs were thawed, selected and activated with CD3/CD28 beads, and expanded in the presence of IL2. The patient was admitted for cyclophosphamide and fludarabine (300 mg/m2 and 30 mg/m2, respectively, ×3 consecutive days) for lymphodepletion prior to the administration of CAR T cells. This was a 3×3 dose escalation study, and the patient presented was treated on dose level 3, at a planned 450×10⁶ dose of CAR⁺ viable T cells.

Radiation therapy: External beam radiation therapy using 6MV photons was delivered using a linear accelerator (Varian Medical Systems, Palo Alto, Calif.). The target areas included the T1 to T8 vertebral bodies with a right-sided paraspinal tumor mass and the whole brain to the C2 vertebral body). Conventional opposed fields were used (AP/PA for the thorax and opposed laterals for the whole brain fields). The total dose was 2000 cGy in 5 daily fractions to each site.

Cytokine measurement: IL-1β, IL6, IL-10, and TNFα in peripheral serum were detected by 4-plex microfluidic sandwich immunoassays performed on an Ella (Protein Simple; San Jose, Calif.). CRP was detected by automated immunoturbidimetric assay performed on an Abbott Architect Chemistry Analyzer (Abbott Laboratories; Chicago, Ill.). Ferritin was detected by automated two-site immunoenzymatic assay on a Tosoh AIA 2000 (Tosoh Bioscience; Japan). D-dimer was detected by automated immunoturbidimetric assay on the Stago STA-R Max analyzer (Stago; France).

Flow cytometry: To stain for CAR T cells, anti-CD3 clone 7D-6 (Thermo-Fisher; Waltham, Mass.), anti-CD8 clone 3B5 (Thermo-Fisher; Waltham, Mass.), and cetuximab (Eli Lilly, Indianapolis, Ind.) conjugated with a lightning link labeling kit (Innova Biosciences, Cambridge, UK) were used. Viability was ascertained with 7AAD exclusion (Thermo-Fisher; Waltham, Mass.). Flow cytometry was performed on a BD LSR II (BD biosciences; San Jose, Calif.), and analyzed with FlowJo (FlowJo LLC; Ashland, Oreg.).

TCR clonotype tracking: DNA was extracted via QIAmp Blood DNA mini kit (Qiagen; Hilden, G R) from PBMCs or bone marrow mononuclear cells. Clonotypes were monitored by TCR Vβ CDR3 sequencing via immunoSEQ assay (Adaptive Biotechnologies; Seattle, Wash.).

### Results

### Clinical Evaluation

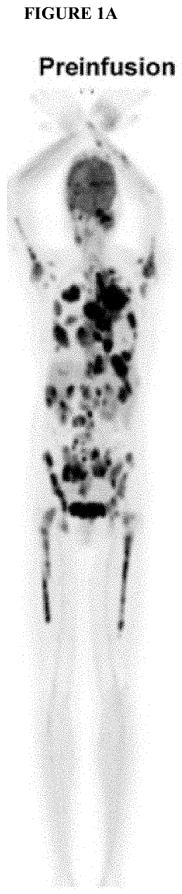

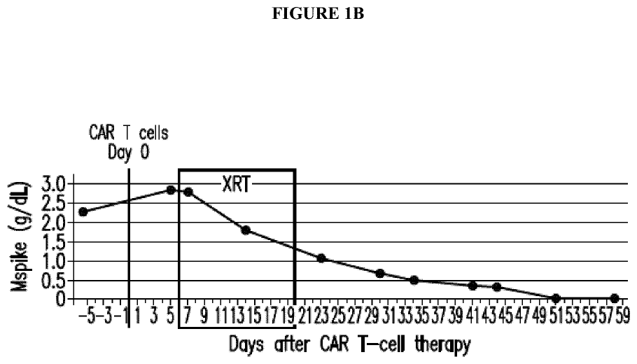

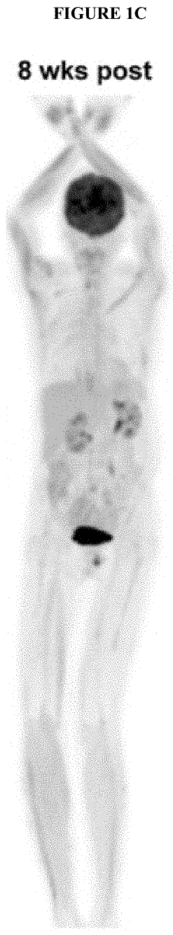

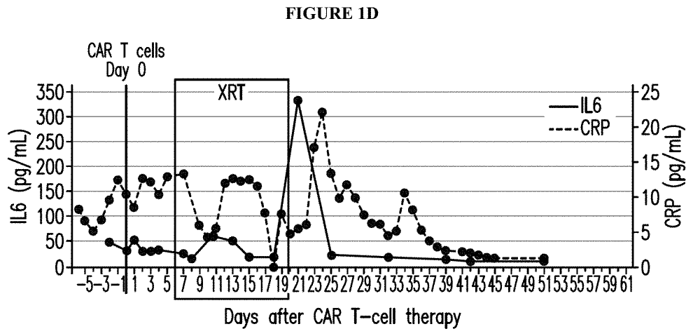

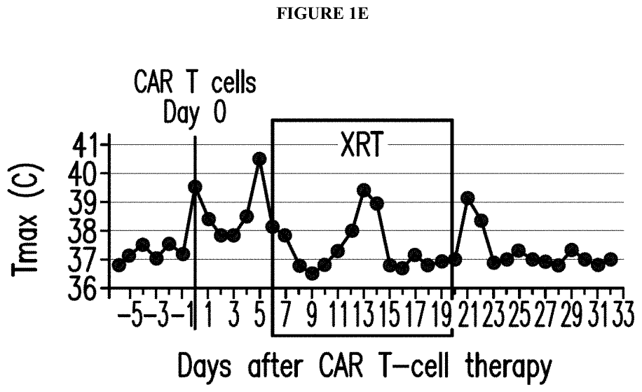

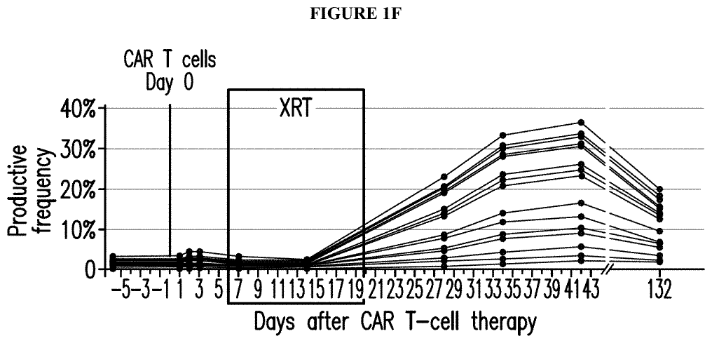

A 63-year-old African American woman was diagnosed with IgAλ multiple myeloma (MM) with high-risk cytogenetics (amplification 1q21) in 2010. She received 8 lines of therapy including combinations with lenalidomide, bortezomib, pomalidomide, carfilzomib, daratumumab, and autologous stem cell transplant, each time with progression. Most recently her disease was refractory to therapy with dexamethasone, cyclophosphamide, etoposide, and cisplatin (DCEP). She enrolled on a clinical trial of BCMA-targeted CAR T-cell therapy including a 4-1BB co-stimulatory domain (Smith et al., Mol Ther. (2018 Jun. 6); 26(6):1447-1456) (NCT03070327). Her clinical course is presented in FIGS. 1A-1F. Baseline findings indicated extensive disease, including monoclonal paraprotein (M-spike) of 2.26 g/dL, a baseline bone marrow biopsy revealing 95% plasma cell infiltration, and a PET/CT scan demonstrating widespread bone-based and extra-osseous MM lesions, including extensive soft tissue and pleural-based masses (see FIG. 1A).

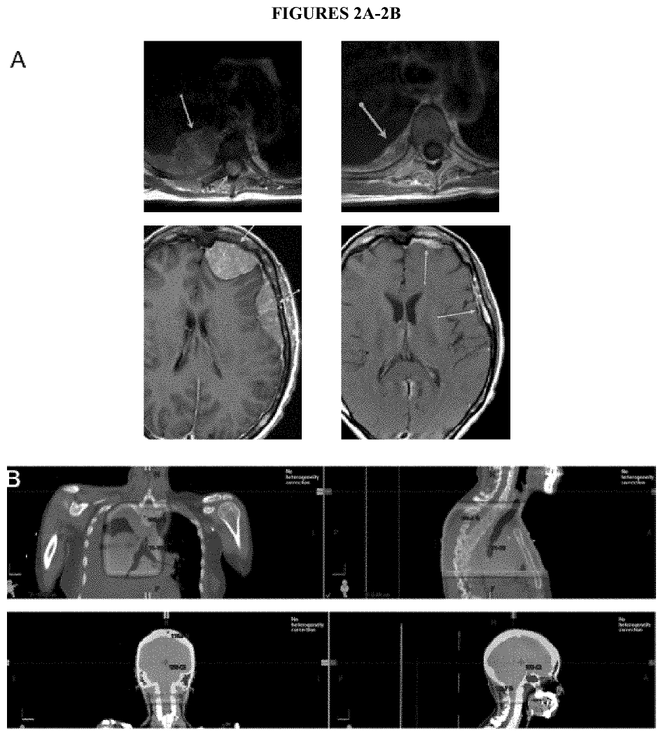

The patient received cyclophosphamide/fludarabine lymphodepleting chemotherapy followed by a single infusion of CAR T cells. Shortly thereafter, she exhibited signs of lower extremity weakness and confusion. The patient remained awake and alert, however, mental status changes in addition to confusion manifested as intermittent aphasia, decreased short-term recall (could recall 0/3 objects), and attention/calculation (could not spell WORLD backwards). To evaluate these neurologic findings an MM spine and brain were obtained which revealed spinal cord compression and multiple epidural masses causing a cerebral mid-line shift (FIG. 2A). On day 5 post-CAR T cells, M-spike had increased from baseline to 2.83 g/dL. Although her neurologic symptoms resolved by this point, given the lesion compressing her thoracic spinal cord on imaging and increasing MM serologic markers, there was concern for progression of her cord compression. The patient therefore received high-dose steroids (dexamethasone taper starting at 10 mg every 12 hours for a total of 13 days) and palliative XRT to the thoracic spine (T1 to T8) followed by the whole brain to C2; delivered between day 6-20 post-CAR T cells (2000 cGy in 5 fractions to each site, in series) (see FIGS. 1A-1F and 2B). Her Mill at 4 weeks showed near-resolution of disease at the sites of radiation therapy (see FIG. 2A).

In addition to this response at the site of radiation therapy, a favorable systemic response was subsequently observed. Seven weeks post-CAR T cells, her M-spike was undetectable (FIG. 1B). PET/CT at 8 weeks post-CAR T cells showed complete radiographic resolution of disease including innumerable sites outside the radiation field (FIG. 1C). The patient's clinical response persisted through 9 months post-therapy.

### Inflammatory Response

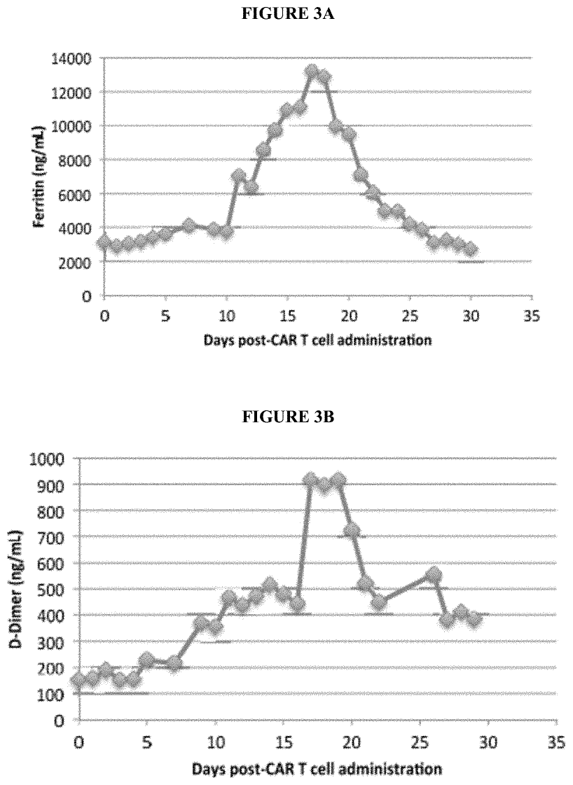

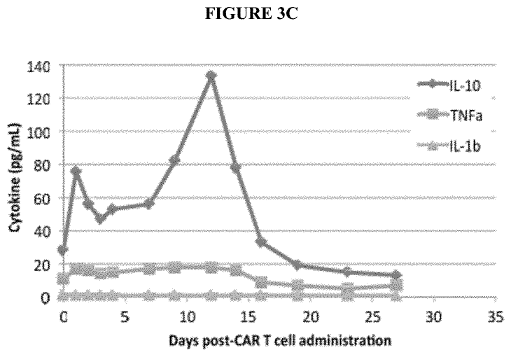

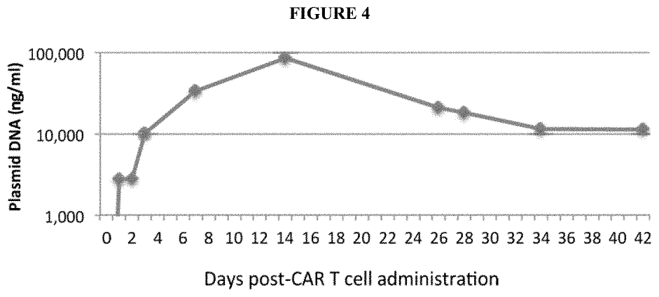

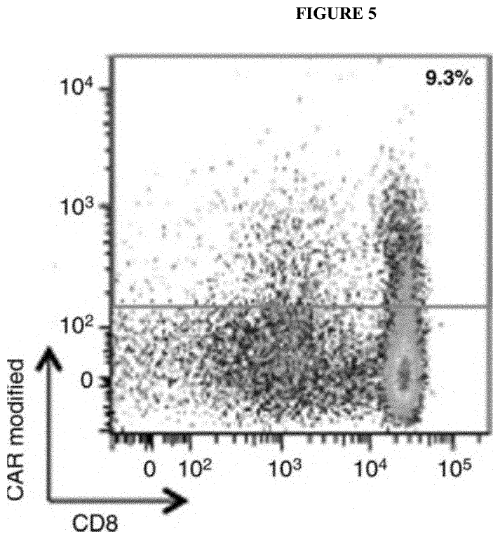

Changes in serum inflammatory markers CRP, Ferritin, and D-dimer, as well as cytokines IL-6, IL-10, TNFα, and IL-10, were prospectively monitored. Inflammatory markers associated with CAR T-cell activity (Davila et al., Sci Transl Med (2014); 6:224ra25) increased shortly after the conclusion of radiation therapy. These included markers characteristic of CAR T cell mediated cytokine release syndrome (CRS) (17): IL-6 (to 333 pg/mL from 32 pg/mL) (see FIG. 1D) and CRP (to 22.11 mg/dL from 6.43 mg/dL) (see FIG. 1D), as well as more general markers of inflammation that are often seen elevated in CRS: Ferritin (to 13,197 ng/mL from 2,921 ng/mL (see FIG. 3A) and D-Dimer (to 915 ng/mL from <150 ng/mL) (see FIG. 3B). Serum IL-10 had a bi-modal peak. The first peak occurred early after the administration of CAR T cells, corresponding to timing of peak serum IL-10 concentrations after CAR T cells reported by others (Kochenderfer et al., J Clin Oncol (2017); 35:1803-13) and the timing of CRS seen in other patients on this study (Mailankody et al., American Society of Hematology (2018); 132:959-959). A later second peak occurred during radiation therapy (see FIG. 3C). TNFα and IL-10 remained stable and within normal limits throughout the clinical course (see FIG. 3C). Around the conclusion of radiation therapy, the patient developed a fever to >39° C. with no clinical or laboratory evidence of infection (see FIG. 1E). At this time, she became tachycardic to 110 s beats/min (from recent baseline low 60 s beats/min) and SBP decreased to 90 s mmHg (from recent baseline 110-130 s mmHg); HR and BP returned to baseline after 3 days of tachycardia and relative hypotension. She did not require vasopressors or supplemental oxygen, therefore this would be considered grade 1 CRS. A delayed CRS-like response was only seen in this patient and not in other patients on the trial (n=13). Despite 13 days of systemic steroids, CART cells expanded and persisted (see FIG. 4), representing 9.3% of CD3⁺ cells in the blood by flow cytometry around the time of inflammatory response (see FIG. 5). Given the temporal relationship of these CRS-like findings immediately after radiation therapy, the timing of changes in her T-cell receptor (TCR) repertoire was investigated using stored samples.

### TCR Clonotype Serial Analysis

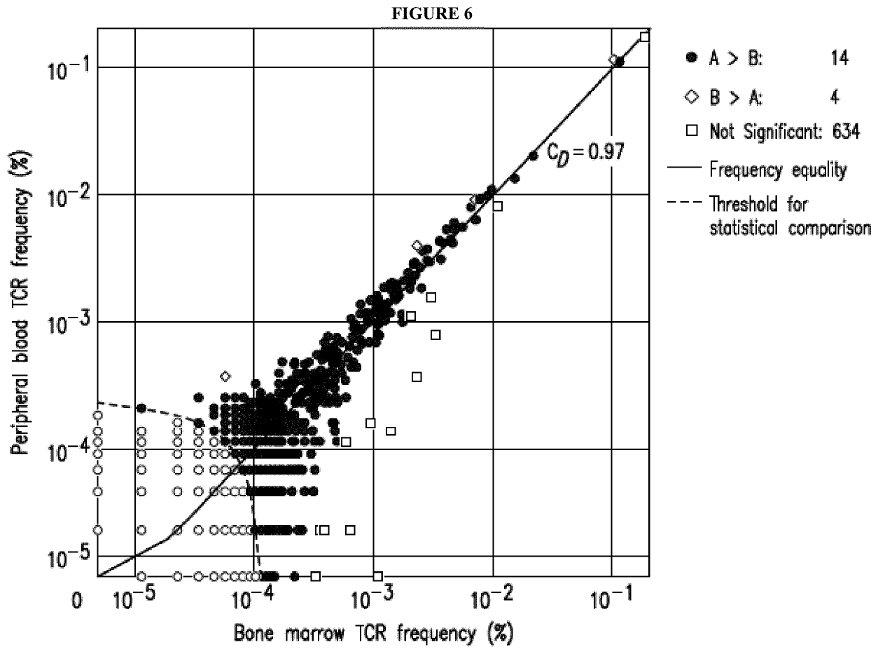

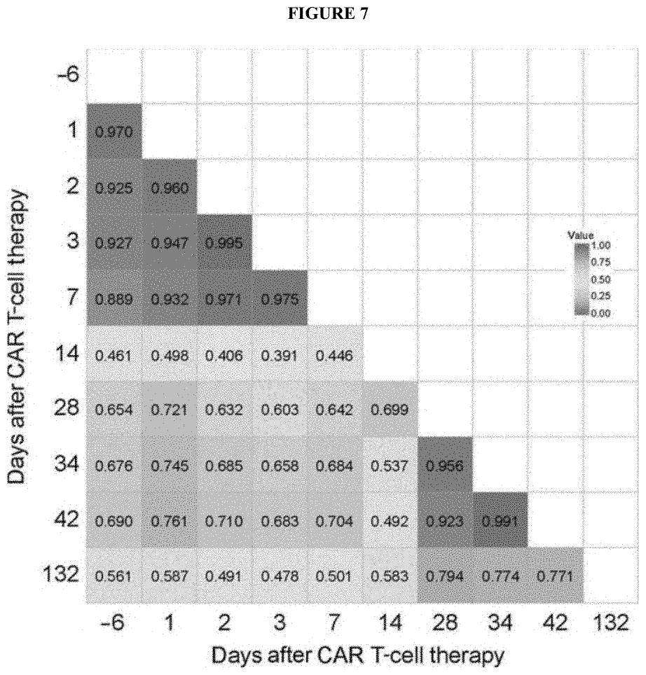

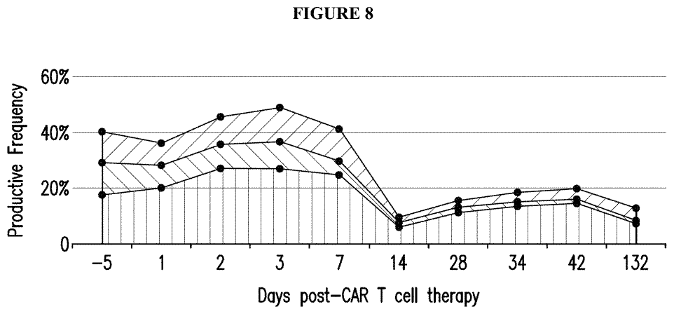

Because the post-radiation findings were suggestive of clinical and laboratory findings often correlating with cytokine release syndrome (CRS), the timing of changes in the patient's T-cell receptor (TCR) repertoire was investigated using stored samples. It was found that the patient's baseline peripheral blood TCR diversity mirrored that in her baseline bone marrow (see FIG. 6; TCR diversity compared by Morisita's Overlap Index; CD=0.97). Given this high degree of overlap between peripheral blood and bone marrow, it was concluded that the peripheral blood may be a suitable surrogate to investigate changes in TCR diversity over time in this patient with intra- and extra-osseous MM. Serial analysis of the patient's peripheral blood TCR repertoire demonstrated TCR diversity was stable at four time points across the first week after CAR T-cell infusion. Values for CD compared to baseline were 0.89-0.97 throughout this time (see FIG. 7). A spike in new TCR clone expansion was noted upon initial evaluation, day eight after the conclusion of radiation therapy (C_{D}=0.56-0.69, 4-19 weeks post-CAR T-cell therapy vs baseline). These newly expanded clones made up >30% of the T-cell repertoire at 4 weeks. Many persisted through at least 19 weeks post CAR T-cell infusion, the most recent time point assessed (see FIG. 1F). No TCR clone expansion was seen in any clones highly expressed at baseline (see FIG. 8).

### DISCUSSION

This Example reports a patient receiving CAR T-cell therapy for refractory MM who received urgent high-dose steroids and palliative XRT to the whole brain and thoracic spine days later. The results illustrate how BCMA-targeted CAR T-cell therapy can facilitate elimination of a large MM burden (including substantial extra-osseous disease) despite the early and continued administration of high-dose steroids. Furthermore, the timing of CRS-like clinical signs and inflammatory markers coincided with the expansion of new TCR clones after radiation therapy, supporting a synergistic effect between XRT and CAR T-cell therapy. The patient's clinical course suggests that local XRT in combination with CAR T-cell therapy may enhance a systemic anti-tumor effect.

For frequent, serial assessment of TCR clonality, peripheral blood mononuclear cells were relied. Despite the fact that MM is a bone marrow based malignancy, and although marrow is accessible, bone marrow cannot be sampled as frequently as can peripheral blood. It was found that, when assessed at the same time point, the patient's bone marrow mononuclear cells and PBMCs had a high degree of overlap in TCR repertoires (C_{D}=0.97). Therefore, it was concluded that TCR clonality in the peripheral blood is relevant in this patient with widespread MM.

Serial analysis of TCR repertoires revealed expansion of new TCR clones coinciding with clinical and laboratory signs of CRS. The confluence of these events shortly after radiation therapy in this previously CAR T-cell treated patient supports the likelihood of synergy between radiation and CAR T-cell therapy.

One limitation of this study is that the stored material was not of sufficient quantity to determine the antigen targets driving the specific TCR clone expansion. However, newly expanded TCR clones comprised >30% of the T-cell population after XRT at a time when CAR′ T cells constituted ˜10% of CD3 cells, indicating that at least some T-cell expansion was driven by non-CAR modified T cells.

Radiation therapy, alone or in combination with checkpoint blockade, is known to “shape” the TCR repertoire of expanded clones (Sridharan Br J Cancer (2016); 115:252-60; Twyman-Saint et al., Nature (2015); 520:373-7). However, given that CARs result in HLA-independent T-cell activation, it is not necessarily intuitive that local radiation therapy would buoy systemic responses by CAR T cells in the way that it can buoy checkpoint blockade, i.e. through increased neo-antigen exposure through an abscopal effect (Postow et al., N Engl J Med. (2012); 366:925-31).

Synergy of CAR T-cell and radiation therapy may occur through several possible mechanisms: (1) Synergy could be explained by the cytokines secreted by CAR T-cells, increasing the likelihood of endogenous T-cells mounting an abscopal-like response. (2) As has been shown pre-clinically, radiation therapy may enhance effector functions and migration of CAR T-cells (Weiss et al., Clin Cancer Res (2018); 24:882-95; DeSelm et al., Mol Ther (2018); 26:2542-52). (3) Increased signaling through the TCR of CAR T-cells may enhance clonal CAR T-cell expansion. Combinations of these mechanisms or unknown mechanisms are also possible explanations.

A similar phenomenon may have been at work in a CAR T cell-treated patient with CNS lymphoma for whom, after relapse, biopsy of a mass precipitated a clinical remission (Abramson et al., N Engl J Med (2017); 377:783-4). These observations support further investigation of CAR T-cell therapy combined with radiation therapy.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of treatment, comprising:
(a) administering to a subject having a disease or condition a dose of cells expressing a recombinant receptor that binds to an antigen; and
(b) administering to said subject radiation, wherein initiation of administration of the radiation is no later than about two weeks after administration of the recombinant receptor-expressing cells.

2. The method of claim 1, wherein initiation of administration of the radiation is no later than about one week after administration of the recombinant receptor-expressing cells.

3. The method of claim 1, wherein initiation of administration of the radiation is between about 5 days and about 10 days after administration of the recombinant receptor-expressing cells.

4. The method of claim 1, wherein the subject has not relapsed at the time of or immediately prior to initiation of administration of the radiation.

5. The method of claim 1, wherein the disease or condition is a tumor or a cancer.

6. The method of claim 1, wherein the disease or condition is selected from the group consisting of blood cancers, B cell malignancies, colon cancer, lung cancer, liver cancer, breast cancer, prostate cancer, ovarian cancer, skin cancer, melanoma, bone cancer, brain cancer, ovarian cancer, epithelial cancers, renal cell carcinoma, pancreatic adenocarcinoma, cervical carcinoma, colorectal cancer, glioblastoma, neuroblastoma, Ewing sarcoma, medulloblastoma, osteosarcoma, synovial sarcoma, mesothelioma, and combinations thereof.

7. The method of claim 6, wherein the blood cancer is selected from the group consisting of leukemia, lymphoma, chronic lymphocytic leukemia (CLL), acute-lymphoblastic leukemia (ALL), Hodgkin Lymphoma, non-Hodgkin's lymphoma, Waldenstrom's Macroglobulinemia, acute myeloid leukemia, multiple myeloma, mantle cell lymphoma, and indolent B cell lymphoma.

8. The method of claim 6, wherein the disease or condition is multiple myeloma.

9. The method of claim 1, wherein the antigen is a tumor antigen.

10. The method of claim 9, wherein the tumor antigen is selected from the group consisting of BCMA, GPRC5D, FcRL5, orphan tyrosine kinase receptor ROR1, tEGFR, Her2, L1-CAM, CD19, CD20, CD22, mesothelin, CEA, epatitis B surface antigen, anti-folate receptor, CD23, CD24, CD30, CD33, CD38, CD44, EGFR, EGP-2, EGP-4, OEPHa2, Erb-B2, Erb-B3, Erb-B4, FBP, fetal acetylcholine receptor (AChR), GD2, GD3, HMW-MAA, IL-22R-alpha, IL-13R-alpha2, KDR, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, MAGE A3, CE7, Wilms Tumor 1 (WT-1), a cyclin, and biotinylated molecules.

11. The method of claim 10, wherein the tumor antigen is BCMA.

12. The method of claim 1, wherein the recombinant receptor is a T cell receptor (TCR) or a functional non-T cell receptor.

13. The method of claim 12, wherein the recombinant receptor is a chimeric antigen receptor (CAR).

14. The method of claim 13, wherein the CAR comprises an extracellular antigen-binding domain that specifically binds to the antigen and an intracellular signaling domain.

15. The method of claim 14, wherein the intracellular signaling domain comprises an intracellular domain of a CD3-zeta (CD3) chain.

16. The method of claim 14, wherein the intracellular signaling domain further comprises a costimulatory signaling region.

17. The method of claim 16, wherein the costimulatory signaling region comprises a signaling domain of CD28 or a portion thereof, a signaling domain of 4-1BB or a portion thereof, a signaling domain of OX40 or a portion thereof, a signaling domain of ICOS or a portion thereof, a signaling domain of DAP-10 or a portion thereof, or a combination thereof

18. The method of claim 17, wherein the costimulatory signaling region comprises a signaling domain of 4-1BB or a portion thereof.

19. The method of claim 13, wherein the CAR further comprises a transmembrane domain.

20. The method of claim 19, wherein the transmembrane domain comprises a transmembrane domain of CD8 or a portion thereof, or a transmembrane domain of CD28 or a portion thereof.

21. The method of claim 20, wherein the transmembrane domain comprises a transmembrane domain of CD8 or a portion thereof.

22. The method of claim 14, wherein the extracellular antigen-binding domain comprises a scFv.

23. The method of claim 14, wherein the extracellular antigen-binding domain comprises: a heavy chain variable region (“V_{H}”) CDR1 comprising the amino acid sequence set forth in SEQ ID NO: 1; a V_{H} CDR2 comprising the amino acid sequence set forth in SEQ ID NO: 2; a V_{H} CDR3 comprising the amino acid sequence set forth in SEQ ID NO: 3; a light chain variable region (“V_{L}”) CDR1 comprising the amino acid sequence set forth in SEQ ID NO: 4; a V_{L} CDR2 comprising the amino acid sequence set forth in SEQ ID NO: 5; and a V_{L} CDR3 comprising the amino acid sequence set forth in SEQ ID NO: 6.

24. The method of claim 1, wherein the cell is a T cell.

25. The method of claim 24, wherein the T cell is selected from the group consisting of a cytotoxic T lymphocyte (CTL), a regulatory T cell, a tumor-infiltrating lymphocyte (TIL), and a Natural Killer T (NKT) cell.

26. The method of claim 1, wherein the cell is autologous or allogenic to the subject.

27. The method of claim 1, wherein the dose of cells comprises cells in an amount sufficient for reduction in burden of a disease or condition in the subject.

28. The method of claim 1, comprising administering to the subject a consecutive dose of the recombinant receptor-expressing cells after administration of a first dose of the recombinant receptor-expressing cells.

29. The method of claim 1, wherein the radiation is selected from the group consisting of external beam radiation, a radiopharmaceutical agent, and a combination thereof.

30. The method of claim 1, wherein the radiation is external beam radiation.

31. The method of claim 1, wherein a total of at least about 10 Gy of radiation is administered to a lesion site of the subject.

32. The method of claim 31, wherein a total of between about 10 Gy and about 30 Gy of radiation is administered to a lesion site of the subject.

33. The method of claim 32, wherein a total of about 20 Gy of radiation is administered to a lesion site of the subject.

34. The method of claim 1, wherein administration of the radiation and administration of the recombinant receptor-expressing cells provide a synergistic abscopal effect, delayed or reduced CRS-like response, and/or systemic expansion of new T-cell receptor (TCR) clone.
